SEQUENCE LISTING (34 sequences)

SEQ ID NO: 1 - RNA, 25 nt, synthetic construct
  source 1..25: mol_type = other RNA
cctcctcgcctggttctggaagaca

SEQ ID NO: 2 - RNA, 20 nt, synthetic construct
  source 1..20: mol_type = other RNA
ttggcccctcctcgcctggt

SEQ ID NO: 3 - RNA, 20 nt, synthetic construct
  source 1..20: mol_type = other RNA
ctgcagttggcccctcctcg

SEQ ID NO: 4 - RNA, 20 nt, synthetic construct
  source 1..20: mol_type = other RNA
tcaaacccctcacctgcagt

SEQ ID NO: 5 - RNA, 25 nt, synthetic construct
  source 1..25: mol_type = other RNA
cctgcagttggcccctcctcgcctg

SEQ ID NO: 6 - RNA, 25 nt, synthetic construct
  source 1..25: mol_type = other RNA
cccctcacctgcagttggcccctcc

SEQ ID NO: 7 - RNA, 17 nt, synthetic construct
  source 1..17: mol_type = other RNA
gcagttggcccctcctc

SEQ ID NO: 8 - RNA, 25 nt, synthetic construct
  source 1..25: mol_type = other RNA
cacctgcagttggcccctcctcgcc

SEQ ID NO: 9 - RNA, 25 nt, synthetic construct
  source 1..25: mol_type = other RNA
atcctcccacagagcctgtacggag

SEQ ID NO: 10 - RNA, 25 nt, synthetic construct
  source 1..25: mol_type = other RNA
ccctgatcctcccacagagcctgta

SEQ ID NO: 11 - RNA, 25 nt, synthetic construct
  source 1..25: mol_type = other RNA
ggcgttgccgcctttgccacaagat

SEQ ID NO: 12 - RNA, 25 nt, synthetic construct
  source 1..25: mol_type = other RNA
ctcacctctcctctcctcggcgttg

SEQ ID NO: 13 - RNA, 25 nt, synthetic construct
  source 1..25: mol_type = other RNA
ctccactcacctctcctctcctcgg

SEQ ID NO: 14 - RNA, 20 nt, synthetic construct
  source 1..20: mol_type = other RNA
agcagggcggctgtccccag

SEQ ID NO: 15 - RNA, 25 nt, synthetic construct
  source 1..25: mol_type = other RNA
gccacaagatgaccccaatgagcag

SEQ ID NO: 16 - RNA, 25 nt, synthetic construct
  source 1..25: mol_type = other RNA
cctttgccacaagatgaccccaatg

SEQ ID NO: 17 - RNA, 25 nt, synthetic construct
  source 1..25: mol_type = other RNA
tgccgcctttgccacaagatgaccc

SEQ ID NO: 18 - RNA, 25 nt, synthetic construct
  source 1..25: mol_type = other RNA
tcctcggcgttgccgcctttgccac

SEQ ID NO: 19 - RNA, 13 nt, synthetic construct
  source 1..13: mol_type = other RNA
tttgccacaagat

SEQ ID NO: 20 - RNA, 20 nt, synthetic construct
  source 1..20: mol_type = other RNA
ccgcctttgccacaagatga

SEQ ID NO: 21 - RNA, 25 nt, synthetic construct
  source 1..25: mol_type = other RNA
tttcttgttgaccatccccccagtc

SEQ ID NO: 22 - RNA, 25 nt, synthetic construct
  source 1..25: mol_type = other RNA
ccatccccccagtcacatgtgttgg

SEQ ID NO: 23 - RNA, 25 nt, synthetic construct
  source 1..25: mol_type = other RNA
cctcctcgcctggttctggaagaca

SEQ ID NO: 24 - RNA, 20 nt, synthetic construct
  source 1..20: mol_type = other RNA
ctggcccctcatcgccggtt

SEQ ID NO: 25 - RNA, 20 nt, synthetic construct
  source 1..20: mol_type = other RNA
cttcagctggcccctcatcg

SEQ ID NO: 26 - RNA, 20 nt, synthetic construct
  source 1..20: mol_type = other RNA
tccagtccctcaccttcagc

SEQ ID NO: 27 - RNA, 25 nt, synthetic construct
  source 1..25: mol_type = other RNA
tgggttgtcgttttcgccacaggat

SEQ ID NO: 28 - RNA, 23 nt, synthetic construct
  source 1..23: mol_type = other RNA
actactcccaggcctcccaggat

SEQ ID NO: 29 - RNA, 22 nt, synthetic construct
  source 1..22: mol_type = other RNA
gcagggctactactcccaggca

SEQ ID NO: 30 - RNA, 25 nt, synthetic construct
  source 1..25: mol_type = other RNA
tctcactcacctctcctcacgcctg

SEQ ID NO: 31 - RNA, 25 nt, synthetic construct
  source 1..25: mol_type = other RNA
cccagactcacccacagagcctgta

SEQ ID NO: 32 - RNA, 127 nt, Homo sapiens
  source 1..127: mol_type = mRNA
gctctgtgggaggatcagggctgggaactctagccctggccctggggatcctgggaggcctggggacagccgccctgctcattggggtcatcttgtggcaaaggcggcaacgccgaggagaggagag

SEQ ID NO: 33 - RNA, 17 nt, synthetic construct
  source 1..17: mol_type = other RNA
gcagttggcccctcctc

SEQ ID NO: 34 - RNA, 18 nt, Synthetic construct
  source 1..18: mol_type = other RNA
tcgttttcgccacaggat

(19) United States
(12) Patent Application Publication
(10) Pub. No.: US 2026/0286366 A1
(43) Pub. Date: Sep. 24, 2026

(54) ADMINISTRATION OF OLIGONUCLEOTIDES

(71) Applicant: RAGE BIOTECH PTY LTD, Melbourne, Victoria (AU)

(72) Inventors: Merlin Christopher Thomas, Melbourne (AU); Raelene Jane Pickering, Melbourne (AU)

(73) Assignee: RAGE BIOTECH PTY LTD, Melbourne, Victoria (AU)

(21) Appl. No.: 19/480,309

(22) PCT Filed: May 3, 2024
(86) PCT No.: PCT/AU2024/050433

§ 371 Date: Oct. 30, 2025

(30) Foreign Application Priority Data
May 5, 2023 (AU) 2023901365
Oct. 5, 2023 (WO) PCT/AU2023/050961

(51) Int. Cl. C12N 15/113

(52) U.S. Cl. CPC C12N 15/1138; C12N 2310/11; C12N 2310/315; C12N 2310/3181; C12N 2310/321; C12N 2310/3231; C12N 2310/3233; C12N 2320/32

(57) ABSTRACT

The present invention relates to methods for the modulation of alternative splicing of pre-mRNA coding for the Receptor for Advanced Glycation End-products (RAGE) or part thereof, using antisense oligonucleotides (AONs) to modify the expression and/or activity of RAGE isoforms. In one aspect, the present invention provides a method for promoting the production of endogenous soluble RAGE and/or reducing the production of membrane bound RAGE (mRAGE) in a subject, the method comprising: in a first administration, administering to the respiratory tract of the subject an antisense oligonucleotide (AON) that promotes the production of endogenous soluble RAGE and/or reduces the production of membrane bound RAGE (mRAGE), in a second administration, administering to the respiratory tract of the subject an AON that promotes the production of endogenous soluble RAGE and/or reduces the production of membrane bound RAGE (mRAGE), wherein the second administration is at least about 7 days after the first administration, thereby promoting the production of endogenous soluble RAGE and/or reducing the production of membrane bound RAGE (mRAGE) in the subject.

### FIELD OF THE INVENTION

The present invention relates to methods for the modulation of alternative splicing of pre-mRNA coding for the Receptor for Advanced Glycation End-products (RAGE) or part thereof, using antisense oligonucleotides (AONs) to modify the expression and/or activity of RAGE isoforms, and to methods of treatment of RAGE-related disorders using said modulators.

### RELATED APPLICATION

This application claims the benefit of priority from Australian provisional application no. 2023901365 filed 5 May 2023, and International Application no. PCT/AU2023/050961 filed 5 Oct. 2023, the entire disclosures of which is incorporated herein by reference.

### BACKGROUND OF THE INVENTION

The Receptor for Advanced Glycation End-products (RAGE) is a multivalent type I transmembrane glycoprotein belonging to the immunoglobulin (Ig) superfamily. The human RAGE (AGER) gene lies within the major histocompatibility complex class III region on chromosome 6. It comprises 11 exons and 10 introns, and a 5′ flanking region that regulates its transcription. The transcribed RAGE mRNA is ~1.4 kb, with a short 3′UTR.

The 50-55 kDa glycosylated RAGE protein is constitutively expressed in a limited range of cells (e.g. vascular endothelium, type I pneumocytes, leukocytes), although RAGE expression may be induced in most cell types and tissues following injury, stress, hypoxia or inflammation, providing a conduit for pro-inflammatory and pro-proliferative signalling. RAGE expression is consequently upregulated in inflammatory and metabolic disorders including but not limited to neurodegenerative disease, cancer, cardiovascular disease, diabetes, autoimmune and ischaemic injury in which RAGE is also implicated in the development and progression.

Under healthy conditions, the lungs' expression of RAGE is the highest of all tissues. Upregulation of RAGE signalling in the lung in other cells and at other sites has been implicated in a range of lung disorders including: chronic obstructive pulmonary disease (COPD)/emphysema; asthma; injury due to cigarette smoking/pollution; acute lung injury/Acute Respiratory Distress Syndrome; and pulmonary fibrosis.

The alternative splicing of RAGE is also important for the regulation of RAGE activity, through the generation of RAGE isoforms that have an altered ability to be activated by ligand-dependent and ligand-independent signalling pathways. The alternative splicing of RAGE is altered in disease states including malignancy, diabetes and Alzheimer's disease.

While alternative splicing appears to be important for RAGE regulation/dysregulation, there is a need for new or improved methods to target this mechanism.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

In one aspect, the present invention provides a method for promoting the production of endogenous soluble RAGE and/or reducing the production of membrane bound RAGE (mRAGE) in a subject, the method comprising:
in a first administration, administering to the respiratory tract of the subject an antisense oligonucleotide (AON) that promotes the production of endogenous soluble RAGE and/or reduces the production of membrane bound RAGE (mRAGE),
in a second administration, administering to the respiratory tract of the subject an AON that promotes the production of endogenous soluble RAGE and/or reduces the production of membrane bound RAGE (mRAGE),
wherein the second administration is at least about 7 days after the first administration,
thereby promoting the production of endogenous soluble RAGE and/or reducing the production of membrane bound RAGE (mRAGE) in the subject.

In another aspect, the present invention provides a method for preventing or treating a disease associated with, or caused by, RAGE in a subject, the method comprising:
in a first administration, administering to the respiratory tract of the subject an antisense oligonucleotide (AON) that that promotes the production of endogenous soluble RAGE and/or reduces the production of membrane bound RAGE (mRAGE),
in a second administration, administering to the respiratory tract of the subject an AON that promotes the production of endogenous soluble RAGE and/or reduces the production of membrane bound RAGE (mRAGE),
wherein the second administration is at least about 7 days after the first administration,
thereby preventing or treating a disease associated with, or caused by, RAGE in a subject.

In one aspect, the present invention provides a method for promoting the production of endogenous soluble RAGE and/or reducing the production of membrane bound RAGE (mRAGE) in a subject, the method comprising:
in a first administration, administering to the respiratory tract of the subject an antisense oligonucleotide (AON) that promotes the production of endogenous soluble RAGE and/or reduces the production of membrane bound RAGE (mRAGE),
in further administrations, administering to the respiratory tract of the subject an AON that promotes the production of endogenous soluble RAGE and/or reduces the production of membrane bound RAGE (mRAGE),
wherein each further administration is at least about 7 days after the previous administration,
thereby promoting the production of endogenous soluble RAGE and/or reducing the production of membrane bound RAGE (mRAGE) in a subject.

In another aspect, the present invention provides a method for preventing or treating a disease associated with, or caused by, RAGE in a subject, the method comprising:
in a first administration, administering to the respiratory tract of the subject an antisense oligonucleotide (AON) that promotes the production of endogenous soluble RAGE and/or reduces the production of membrane bound RAGE (mRAGE),
in further administrations, administering to the respiratory tract of the subject an AON that promotes the production of endogenous soluble RAGE and/or reduces the production of membrane bound RAGE (mRAGE),
wherein each further administration is at least about 7 days after the first administration or previous administration,
thereby preventing or treating a disease associated with, or caused by, RAGE in a subject.

In one aspect, the present invention provides an antisense oligonucleotide (AON) that promotes the production of endogenous soluble RAGE and/or reduces the production of membrane bound RAGE (mRAGE) for use in promoting the production of endogenous soluble RAGE in a subject, the use comprising:
in a first administration, administering the AON to the respiratory tract of the subject,
in a second administration, administering the AON to the respiratory tract of the subject,
wherein the second administration is at least about 7 days after the first administration,
thereby promoting the production of endogenous soluble RAGE and/or reducing the production of membrane bound RAGE (mRAGE) in the subject.

In another aspect, the present invention provides an antisense oligonucleotide (AON) that promotes the production of endogenous soluble RAGE and/or reduces the production of membrane bound RAGE (mRAGE) for use in preventing or treating a disease associated with, or caused by, RAGE in a subject, the use comprising:
in a first administration, administering the AON to the respiratory tract of the subject,
in a second administration, administering the AON to the respiratory tract of the subject,
wherein the second administration is at least about 7 days after the first administration,
thereby preventing or treating a disease associated with, or caused by, RAGE in a subject.

In one aspect, the present invention provides an antisense oligonucleotide (AON) that promotes the production of endogenous soluble RAGE for use in promoting the production of endogenous soluble RAGE and/or reducing the production of membrane bound RAGE (mRAGE) in a subject, the use comprising:
in a first administration, administering the AON to the respiratory tract of the subject,
in further administrations, administering the AON to the respiratory tract of the subject,
wherein each further administration is at least about 7 days after the previous administration,
thereby promoting the production of endogenous soluble RAGE and/or reducing the production of membrane bound RAGE (mRAGE) in a subject.

In another aspect, the present invention provides an antisense oligonucleotide (AON) that promotes the production of endogenous soluble RAGE and/or reduces the production of membrane bound RAGE (mRAGE) for use in preventing or treating a disease associated with, or caused by, RAGE in a subject, the use comprising:
in a first administration, administering the AON to the respiratory tract of the subject,
in further administrations, administering the AON to the respiratory tract of the subject,
wherein each further administration is at least about 7 days after the first administration or previous administration,
thereby preventing or treating a disease associated with, or caused by, RAGE in a subject.

In one aspect, the present invention provides use of an antisense oligonucleotide (AON) that promotes the production of endogenous soluble RAGE and/or reduces the production of membrane bound RAGE (mRAGE) in the manufacture of a medicament for promoting the production of endogenous soluble RAGE and/or reducing the production of membrane bound RAGE (mRAGE) in a subject, wherein the medicament is formulated for a first and second administration to the subject, wherein the second administration is at least about 7 days after the first administration.

In another aspect, the present invention provides use of an antisense oligonucleotide (AON) that promotes the production of endogenous soluble RAGE and/or reduces the production of membrane bound RAGE (mRAGE) in the manufacture of a medicament for preventing or treating a disease associated with, or caused by, RAGE in a subject, wherein the medicament is formulated for a first and second administration to the subject, wherein the second administration is at least about 7 days after the first administration.

In one aspect, the present invention provides use of an antisense oligonucleotide (AON) that promotes the production of endogenous soluble RAGE and/or reduces the production of membrane bound RAGE (mRAGE) in the manufacture of a medicament for promoting the production of endogenous soluble RAGE in a subject, wherein the medicament is formulated for a first and further administrations to the subject, wherein each further administration is at least about 7 days after the previous administration.

In another aspect, the present invention provides use of an antisense oligonucleotide (AON) that promotes the production of endogenous soluble RAGE and/or reduces the production of membrane bound RAGE (mRAGE) in the manufacture of a medicament for preventing or treating a disease associated with, or caused by, RAGE in a subject, wherein the medicament is formulated for a first and further administrations to the subject, wherein each further administration is at least about 7 days after the previous administration.

In any aspect or embodiment, the second or further administration is at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, or at least about 28 days after the first or previous administration. In a particularly preferred embodiment, the second or further administration is at least about 20 days, at least about 21 days, at least about 22 days, at least about 23 days, at least about 24 days, at least about 25 days, at least about 26 days, at least about 27 days, or at least about 28 days after the first or previous administration.

In any aspect or embodiment, the second or further administration is at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, or at least 28 days after the first or previous administration. In a particularly preferred embodiment, the second or further administration is at least 20 days, at least 21 days, at least 22 days, at least 23 days, at least 24 days, at least 25 days, at least 26 days, at least 27 days, or at least 28 days after the first or previous administration.

In any aspect or embodiment, the second or further administration is 8 days, 9 days, 11 days, 12 days, 13 days, 14 days, 15 days, 16 days, 17 days, 18 days, 19 days, 20 days, 21 days, 22 days, 23 days, 24 days, 25 days, 26 days, 27 days, or 28 days after the first or previous administration. In a particularly preferred embodiment, the second or further administration is 20 days, 21 days, 22 days, 23 days, 24 days, 25 days, 26 days, 27 days, or 28 days after the first or previous administration.

In any aspect or embodiment, each further administration is provided at a regular time interval.

In any aspect or embodiment, the further administrations is at least one administration, at least two administrations or at least three administrations.

In one aspect, the present invention provides a method for promoting the production of endogenous soluble RAGE and/or reducing the production of membrane bound RAGE (mRAGE) in a subject, the method comprising:
administering to the respiratory tract of a subject, once every at least about 7 days, an antisense oligonucleotide (AON) that promotes the production of endogenous soluble RAGE and/or reduces the production of membrane bound RAGE (mRAGE),
thereby manipulating splicing in a RAGE pre-mRNA resulting in the skipping of exon 10 in the subject.

In another aspect, the present invention provides a method for preventing or treating a disease associated with, or caused by, RAGE in a subject, the method comprising:
administering to the respiratory tract of a subject, once every at least about 7 days, an antisense oligonucleotide (AON) that promotes the production of endogenous soluble RAGE and/or reduces the production of membrane bound RAGE (mRAGE),
thereby preventing or treating a disease associated with, or caused by, RAGE in a subject.

In one aspect, the present invention provides an antisense oligonucleotide (AON) that promotes the production of endogenous soluble RAGE and/or reduces the production of membrane bound RAGE (mRAGE) for use in promoting the production of endogenous soluble RAGE and/or reducing the production of membrane bound RAGE (mRAGE) in a subject, the use comprising:
administering the AON to the respiratory tract of a subject, once every at least about 7 days,
thereby manipulating splicing in a RAGE pre-mRNA resulting in the skipping of exon 10 in the subject.

In another aspect, the present invention provides an antisense oligonucleotide (AON) that promotes the production of endogenous soluble RAGE and/or reduces the production of membrane bound RAGE (mRAGE) for use in preventing or treating a disease associated with, or caused by, RAGE in a subject, the method comprising:
administering the AON to the respiratory tract of a subject, once every at least about 7 days,
thereby preventing or treating a disease associated with, or caused by, RAGE in a subject.

In one aspect, the present invention provides use of an antisense oligonucleotide (AON) that promotes the production of endogenous soluble RAGE and/or reduces the production of membrane bound RAGE (mRAGE) in the manufacture of a medicament for promoting the production of endogenous soluble RAGE and/or reducing the production of membrane bound RAGE (mRAGE) in a subject, wherein the medicament is formulated for administration to the respiratory tract of a subject, once every at least about 7 days, to manipulate splicing in a RAGE pre-mRNA resulting in the skipping of exon 10 in the subject.

In another aspect, the present invention provides use of an antisense oligonucleotide (AON) that promotes the production of endogenous soluble RAGE and/or reduces the production of membrane bound RAGE (mRAGE) in the manufacture of a medicament for preventing or treating a disease associated with, or caused by, RAGE in a subject, wherein the medicament is formulated for administration to the respiratory tract of a subject, once every at least about 7 days.

In any aspect or embodiment, the method or use comprises administering an AON to the respiratory tract of the subject once every at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, or at least about 28 days after the first administration. In a particularly preferred embodiment, the method or use comprises administering an AON to the respiratory tract of the subject once every at least about 20 days, at least about 21 days, at least about 22 days, at least about 23 days, at least about 24 days, at least about 25 days, at least about 26 days, at least about 27 days, or at least about 28 days after the first administration.

In any aspect or embodiment, the method or use comprises administering and AON to the respiratory tract of the subject once every at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, or at least 28 days after the first administration. In a particularly preferred embodiment, the method or use comprises administering an AON to the respiratory tract of the subject once every at least at least 21 days, at least 22 days, at least 23 days, at least 24 days, at least 25 days, at least 26 days, at least 27 days, or at least 28 days after the first administration.

In any aspect, the method or use comprises administering an AON to the respiratory tract of the subject once every 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, or 28 days after the first administration. In a particularly preferred embodiment, the method or use comprises administering an AON to the respiratory tract of the subject once every 20 days, 21 days, 22 days, 23 days, 24 days, 25 days, 26 days, 27 days, or 28 days after the first administration.

In any aspect or embodiment, the method or use comprises administering an AON to the respiratory tract of the subject in a first and second administration; in a first, second and third administration; or in a first, second, third and fourth administration.

In any aspect or embodiment, the method or use comprises administering an AON the respiratory tract of the subject at a regular time interval.

In any aspect, a method or use of the invention may promote the production of endogenous soluble RAGE and/or reduce the production of membrane bound RAGE (mRAGE) in a subject, particularly the respiratory tract of the subject. In any embodiment, a method of the invention may promote the production of endogenous soluble RAGE and/or reduce the production of membrane bound RAGE (mRAGE) in one or more tissues of the respiratory tract, for example one or more of the tissues of the respiratory tract described herein.

In any aspect, an AON may promote the production of endogenous soluble RAGE and/or reduce the production of membrane bound RAGE (mRAGE) by promoting the inclusion of exon 9b and/or the exclusion (e.g. skipping) of exon 10. Therefore, in any aspect, the AON may promote splicing in the RAGE pre-mRNA resulting in the inclusion of exon 9b and/or skipping of exon 10. For example, the AON may result in an increase of the level of RAGE_v1, RAGE_v6, RAGE_v8, RAGE_v9 RAGE_v10, RAGE_v15, RAGE_v18, and RAGE_v19 mRNA, preferably an increase in the level of RAGE_v1, RAGE_v6, RAGE_v8, RAGE_v9, RAGE_v10, RAGE_v15, RAGE_v18, and RAGE_v19 mRNA in one or more tissues of the respiratory tract.

In any aspect, the AON is administered to the total respiratory tract, the upper respiratory tract or the lower respiratory tract.

As used herein, the upper respiratory tract may include any one or more of the following regions: the nose and nasal passages, paranasal sinuses, the pharynx, and the portion of the larynx above the vocal folds (cords). Typically, the lower respiratory tract includes any one or more of the following regions: the portion of the larynx below the vocal folds, trachea, bronchi and bronchioles. The lungs can be included in the lower respiratory tract and include the respiratory bronchioles, alveolar ducts, alveolar sacs, and alveoli.

In any aspect, the AON is administered as an aerosol, a dry powder or as nasal drops. In any embodiment, the AON may be administered using a nasal spray pump, intranasal installation, intratrachael instillation, metered dose inhaler (MDI), dry powder inhaler (DPI), nebuliser (jet, ultrasonic mesh or vibrating mesh) or soft mist inhaler (SMI).

In any aspect, the AON is administered in a single dose. For example, the first, second or further administration is a single dose administered to the respiratory tract of the individual. Preferably, a single dose is a contiguous administration of the AON.

In any aspect, the AON is administered via the same route of administration in the first, second or further administrations. In one embodiment, the AON is administered as an aerosol in each of the first, second or further administrations.

In any aspect, the same AON is administered in each of the first, second or further administrations.

In any aspect, the AON is an AON of 10 to 50 nucleotides comprising a targeting sequence complementary to a region near or within an intron of the RAGE pre-mRNA. Alternatively, the AON is 10 to 50 nucleotides comprising a targeting sequence complementary or adjacent to a splice site of the RAGE pre-mRNA.

Because factors such as RNA secondary structure, competition between AONs and SR proteins, heterogeneous nuclear ribonucleoproteins (hnRNPs), and/or other elements that make up the spliceosome can affect AONs' action, AONs directed at the crucial acceptor or donor splice sites will not always alter splicing. Consequently, in any aspect of the invention, the AON is 10 to 50 nucleotides comprising a targeting sequence complementary or adjacent to cis-acting RNA elements in the pre-mRNA of RAGE that act as enhancers or silencers, that, when bound by an elements of the splicosome (e.g. protein-splicing factors, uRNA, lncRNA) modulates the splicing of a nearby exon.

In any aspect, the AON is 10 to 50 nucleotides comprising a targeting sequence complementary to RAGE pre-mRNA which modulates secondary structure of said mRNA to influence splice site selection.

In any aspect, the AON is an isolated or purified AON for inducing exclusion (also known as skipping) of one or more exonic sequences in the RAGE gene transcript or part thereof.

In any aspect, the AON is an isolated or purified AON for inducing retention of intronic sequences in the RAGE gene transcript or part thereof.

In any aspect, the AON comprises at least one modified nucleotide. Typically, the AON is chemically-modified to prevent degradation of the pre-mRNA-AON complex, including but not limited to phosphorodiamidate morpholino oligomers (PMO), 2′ O-methyl phosphorothioate oligonucleotides (2OMe), and 2′-O-methoxyethyl phosphorothioate oligonucleotides (2′-MOE), locked nucleic acid (LNA) modified AONs, thermostable twisted intercalating nucleic acid (TINA) and peptide nucleic acids (PNAs).

In any aspect, the AON comprises at least one modified nucleotide selected from the group consisting of: phosphorodiamidate morpholino oligomers (PMO), 2′ O-methyl oligonucleotides (2OMe), 2′-O-methoxyethyl oligonucleotides (2′-MOE), phosphorothioate oligonucleotides, locked nucleic acid (LNA) modified AONs, thermostable twisted intercalating nucleic acid (TINA) and peptide nucleic acids (PNAs).

In any aspect, the AONs may be conjugated to moieties to increase their delivery, including but not limited to cell-penetrating peptides (CPPs), vivo-morpholinos (VMO) or peptide phosphorodiamidate morpholino oligomers (PPMO).

In any aspect, the antisense oligonucleotide comprises, consists essentially of or consists of a nucleotide sequence at least 80%, 81%, 82%, 83%, 84%, 85%, 86%, 87%, 88%, 89%, 90%, 91%, 92%, 93%, 94%, 95%, 96%, 97%, 98%, 99% or 100% complementary to a target region of exon 10 of RAGE pre-mRNA over the entirety of the antisense oligonucleotide. Preferably, the 5′-most nucleotide of the AON is nucleotide position 88 or 114 of exon 10 or is between nucleotide positions 88 to 114 of exon 10. Preferably, the 5′-most nucleotide of the target region is nucleotide position 88 or 114 of exon 10 or is between nucleotide positions 88 to 114 of exon 10.

In any embodiment, the 5′-most nucleotide of the target region is position 88 or 113, or between nucleotide positions 88 to 113 of exon 10 of RAGE pre-mRNA.

In any embodiment, the 5′-most nucleotide of the target region is position 90 or 113, or between nucleotide positions 90 to 113 of exon 10 of RAGE pre-mRNA.

In any embodiment, the 5′-most nucleotide of the target region is position 88 or 108, or between nucleotide positions 88 to 108 of exon 10 of RAGE pre-mRNA.

In any embodiment, the 5′-most nucleotide of the target region is position 90 or 108, or between nucleotide positions 90 to 108 of exon 10 of RAGE pre-mRNA.

In any embodiment, the 5′-most nucleotide of the target region is position 88 or 95, or between nucleotide positions 88 to 95 of exon 10 of RAGE pre-mRNA.

In any embodiment, the target region is from nucleotide position 88 to 137 of exon 10 of RAGE pre-mRNA.

In any embodiment, the target region is from nucleotide position 88 to 107 of exon 10 of RAGE pre-mRNA.

In any embodiment, the target region is from nucleotide position 90 to 102 of exon 10 of RAGE pre-mRNA.

In any embodiment, the target region is from nucleotide position 95 to 119 of exon 10 of RAGE pre-mRNA.

In any embodiment, the target region is between nucleotide positions 108 to 132 of exon 10 of RAGE pre-mRNA.

In any embodiment, the target region is between nucleotide positions 113 to 137 of exon 10 of RAGE pre-mRNA.

In any aspect, the AON may be 8 to 40 nucleotides in length, 15 to 25 nucleotides in length or 18 nucleotides in length.

In any embodiment, the AON is selected from the group comprising the sequences set forth in any of Tables 1a-1d. Preferably, the AON is selected from the list comprising: SEQ ID NO: 1-31, for example, the AON is SEQ ID NO: 11, 18, 19, or 20 or a nucleotide sequence at least 85%, 90% or 95% identical thereto.

In any embodiment, the AON may be selected to be an AON capable of binding to a selected target site, wherein the target site is a putative mRNA splicing site selected from a splice donor site, splice acceptor site, splice enhancer sequences splice silencer sequences or sites that modulate the secondary structure of pre-mRNA. The target site may also include some flanking intronic sequences when the donor or acceptor splice sites are targeted.

More specifically, the AON may be selected from the group comprising of any one or more of SEQ ID NOs: 1-31 and/or the sequences set forth in any of Tables 1a-1d, and combinations or cocktails thereof. More preferably, the AON is SEQ ID NO: 11, 18, 19, or 20. The combination of AONs is preferably a combination of SEQ ID NO: 11 and 10, or SEQ ID NO: 11 and 13. This includes sequences which can hybridise to such sequences under stringent hybridisation conditions, sequences complementary thereto, sequences containing modified bases, modified backbones, and functional truncations or extensions thereof which possess or modulate pre-mRNA processing activity in a RAGE gene transcript.

In certain embodiments, AONs may be 100% complementary to the target sequence, or may include mismatches, e.g., to accommodate variants, as long as a hetero-duplex formed between the oligonucleotide and target sequence is sufficiently stable to withstand the action of cellular nucleases and other modes of degradation which may occur in vivo. Hence, certain oligonucleotides may have about or at least about 70% sequence complementarity, e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence complementarity, between the oligonucleotide and the target sequence.

The methods and uses of the invention extends also to a combination of two or more AONs capable of binding to a selected target to modulate alternative splicing of the RAGE pre-mRNA, including a construct comprising two or more such AONs. The constructs may be used together for a combined AON-based therapy. The combination of AONs is preferably a combination of SEQ ID NO: 11 and 10, or SEQ ID NO: 11 and 13.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1. Division of the mouse lung as described in the Examples herein.

FIG. 2. Overview of the experimental design. Time-dependent pharmacodynamics activity of a single aerosolised dose of ASO2 in mice.

FIG. 3. Mouse weight following administration of aerosolised ASO2. C57BL/6 mice were administered aerosolised saline vehicle, ASO2 (0.3 mg/kg) or ASO2 (3.0 mg/kg) to the lung and mouse weight was monitored for 21 days. Data shows mean±SEM.

FIG. 4. Aerosolised ASO2 administration increases RAGE_v1 expression in the lung. C57BL/6 mice were administered aerosolised saline vehicle, ASO2 (0.3 mg/kg) or ASO2 (3.0 mg/kg) and RAGE_v1 mRNA levels (encoding esRAGE) were measured in the samples collected from the left lung at various time points up to 28 days following administration. Data shows geometric mean±95% Cl.

FIG. 5. Aerosolised ASO2 administration reduces RAGE expression in the lung. C57BL/6 mice were administered aerosolised ASO2 (3.0 mg/kg) and RAGE mRNA expression levels (encoding full length RAGE) were measured in samples collected from the left lung at various time points up to 28 days following administration. Data shows mean±SEM.

FIG. 6. esRAGE production in the lung increases following aerosolised ASO2 administration compared to vehicle. C57BL/6 mice (about 25 g in weight) were administered aerosolised saline vehicle, ASO2 (7.5 μg for a dose of about 0.3 mg/kg) or ASO2 (75 μg for a dose of about 3.0 mg/kg) to the lung and esRAGE protein levels were measure in samples collected from the left lung at various time points up to 28 days following administration. Data shows mean.

FIG. 7. esRAGE levels in plasma increases following aerosolised ASO2 administration compared to vehicle. C57BL/6 mice (about 25 g in weight) were administered aerosolised saline vehicle, ASO2 (7.5 μg for a dose of about 0.3 mg/kg) or ASO2 (75 μg for a dose of about 3.0 mg/kg) to the lung and esRAGE protein levels were measure in collected plasma samples at various time points up to 28 days following administration. Data shows mean.

FIG. 8. Overview of the experimental design. Time-dependent pharmacodynamics effect of a single intratracheal treatment (aerosolised treatment) with AON m79 in male C57BL/6 mice.

FIG. 9. RAGE_v1 mRNA expression in the left lung after a single dose of AON m79 or control oligonucleotide. C57BL/6 mice were intratracheally administered control AON or AON m79 (3.0 mg/kg) and RAGE_v1 mRNA levels (encoding esRAGE) were measured in the samples collected from the left lung at 3 days, 7 days, 14 days and 28 days following administration. Data shows mean±SEM; *p<0.05 vs control oligonucleotide.

FIG. 10. esRAGE protein expression in the left lung after a single dose of AON m79 or control oligonucleotide. C57BL/6 mice were intratracheally administered control AON or AON m79 (3.0 mg/kg) and esRAGE levels were measured in the samples collected from the left lung at, 7 days, 14 days and 28 days following administration. Data shows mean±SEM; * p<0.05 vs control oligonucleotide.

FIG. 11. Plasma esRAGE levels in the circulation after a single dose of ASO m79 or control oligonucleotide. C57BL/6 mice were intratracheally administered control AON or AON m79 (3.0 mg/kg) and esRAGE levels were measured in the samples collected from plasma at 7 days, 14 days and 28 days following administration. Data shows mean±SEM; * p<0.05 vs control oligonucleotide.

FIG. 12. AON m79 drug level in the left lower lobe of the lung as measured by CL-PCR. C57BL/6 mice were intratracheally administered control oligonucleotide or AON m79 (3.0 mg/kg) and AON drug levels were measured by CL-PCR at 3 days, 7 days, 14 days and 28 days following administration. Data shows mean±SEM; * p<0.05 vs control oligonucleotide.

FIG. 13. RAGE_v1 and RAGE mRNA expression in the left lung following weekly dosing of ASO2. (A) C57BL/6 mice were administered aerosolised saline vehicle weekly for 4 weeks (vehicle group), aerosolised saline vehicle weekly for 3 weeks followed by a single dose of aerosolised ASO2 (3.0 mg/kg) on the 4th week (single dose group), or aerosolised ASO2 (3.0 mg/kg) weekly for 4 weeks (4 dose group). Samples from the left lung were collected at 28 days from the first administration and RAGE_v1 mRNA levels (encoding esRAGE) (B) and RAGE mRNA levels (encoding full-length RAGE) (C) were measured. Data is mean±SEM.

### DETAILED DESCRIPTION OF THE EMBODIMENTS

It will be understood that the invention disclosed and defined in this specification extends to all alternative combinations of two or more of the individual features mentioned or evident from the text or drawings. All of these different combinations constitute various alternative aspects of the invention.

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

One skilled in the art will recognize many methods and materials similar or equivalent to those described herein, which could be used in the practice of the present invention. The present invention is in no way limited to the methods and materials described. It will be understood that the invention disclosed and defined in this specification extends to all alternative combinations of two or more of the individual features mentioned or evident from the text or drawings. All of these different combinations constitute various alternative aspects of the invention.

All of the patents and publications referred to herein are incorporated by reference in their entirety.

For purposes of interpreting this specification, terms used in the singular will also include the plural and vice versa.

The inventors have surprisingly identified that administration of an antisense oligonucleotide (AON) that promotes splicing in the RAGE pre-mRNA resulting in the retention of intron 9 (exon 9b) and/or exclusion (e.g. skipping) of exon 10 to the respiratory tract of a subject results in rapid and sustained indication of alternative splicing resulting in the retention of intron 9 (exon 9b) and/or exclusion (e.g. skipping) of exon 10 for at least 14 days and up to and including 28 days. This previously unknown sustained effect shows that a therapeutically effective frequency of administration of an AON that results in skipping of exon 10 to the respiratory tract of a subject is at least 14 days and up to and including 28 days.

### RAGE Pre-mRNA Alternate Splicing

Alternative splicing is recognized as an important layer of post-transcriptional gene regulation for the Receptor for Advanced Glycation End-products (RAGE). Although most RAGE is expressed in its full length isoform, a number of different coding isoforms are generated through alternative splicing (also known as splicoforms), including splicoforms with N-terminal truncations, C-terminal truncations, and splicoforms retaining intronic sequences. These different splicoforms may act as possible regulators of the full-length RAGE receptor either by competitive ligand binding or by displacing the full-length protein from binding partners. Over twenty splicoforms have been identified in different tissues such as lung, liver, kidney, smooth muscle, endothelial cells and brain.

The different RAGE gene splice variants have been named RAGE, RAGE_v1 to RAGE_v19 according to the Human Gene Nomenclature Committee and are described in Hudson et al., (2008) The FASEB Journal, 22:1572-1580, the contents of which are incorporated in its entirety.

As used herein, “endogenous soluble RAGE” refers to a polypeptide that lacks any signalling elements and/or the transmembrane domain of full-length RAGE. Endogenous soluble RAGE may be encoded, and subsequently translated, by an alternatively spliced RAGE pre-mRNA resulting in the inclusion of exon 9b and/or exclusion (eg skipping) of exon 10, resulting in a premature stop and the complete loss of the trans-membrane and cytoplasmic domains. For example, endogenous soluble RAGE may be encoded by one or more of RAGE_v1, RAGE_v6, RAGE_v8, RAGE_v9 RAGE_v10, RAGE_v15, RAGE_v18, and RAGE_v19 mRNA, preferably RAGE_v1. Therefore, it will be appreciated that any AON as described herein that promotes splicing in the RAGE pre-mRNA resulting in the inclusion of exon 9b and/or exclusion (eg skipping) of exon 10 may be used in the methods of the invention to promote the production of “endogenous soluble RAGE” in the respiratory tract and blood of a subject.

For example, a “endogenous soluble RAGE” as described herein may be endogenous secretory RAGE (esRAGE) which is encoded by RAGE_v1 mRNA. esRAGE constitutes ~5% of circulating RAGE in humans. The skipping of exon 10 in esRAGE-type splicing is ascribed to the limitation of intron length in higher eukaryotes. Roughly 45 nucleotides must separate the 5′ splice site and branch point, and the minimum distance between the branch point and 3′ splice site appears to be approximately 18 nucleotides, respectively. Therefore introns shorter than 70 nucleotides are extremely rare in mammals and cannot be spliced out efficiently. When the esRAGE 5′ splice site in intron 9 is selected, the distance between this site and the 3′ splice site that borders exon 10 is 46 nucleotides, which is considerably shorter than the lower limit of the intron length. Therefore, the use of the downstream, esRAGE 5′ splice site of intron 9 and the inclusion of exon 10 would be mutually exclusive. Among the known splice variants analyzed, all variants that used the downstream esRAGE 5′ splice site in intron 9 skipped exon 10; in contrast, all variants that used the upstream RAGE 5′ splice site in intron 9 included exon 10. Thus, the available evidence indicates that the selection of either one of the two alternative 5′ splice sites in intron 9 couples with inclusion or exclusion of exon 10. The means of regulation of this splicing or an external means to modulate has been previously unknown.

### Soluble Splicoforms of Receptor for Advanced Glycation End-Products (RAGE)

Alternative splicing is recognized as an important layer of post-transcriptional gene regulation for the Receptor for Advanced Glycation End-products (RAGE). Although most RAGE is expressed in its full length isoform, a number of different coding isoforms are generated through alternative splicing (also known as splicofoms), including splicoforms with N-terminal truncations, C-terminal truncations, and splicoforms retaining intronic sequences. These different splicoforms may act as possible regulators of the full-length RAGE receptor either by competitive ligand binding or by displacing the full-length protein from binding partners. Over twenty splicoforms have been identified in different tissues such as lung, liver, kidney, smooth muscle, endothelial cells and brain.

The different RAGE gene splice variants have been named RAGE, RAGE_v1 to RAGE_v19 according to the Human Gene Nomenclature Committee and as described in Hudson et al., (2008) The FASEB Journal, 22:1572-1580, the contents of which are incorporated in its entirety. For example, (run on) retention of intron 9 (exon 9b) results in a premature stop and the complete loss of the trans-membrane and cytoplasmic domains generating a C-terminus truncated splicoform. For example, RAGE_v1 is C-terminus truncated splicoform generated by skipping of exon 10 and retention of intron 9 (exon 9b). Alternatively, in another example, an AON as described herein may increase RAGE_v10 mRNA levels in a tissue or sample by promoting splicing in the RAGE pre-mRNA that results in the skipping of exon 10 and exon 11.

The C-terminus truncated splicoforms as described herein lack any signalling elements or the transmembrane domain and translate a c-terminus truncated RAGE, such as endogenous secretory RAGE (esRAGE), that is able to act as a decoy receptor, competing with full length RAGE for ligands or increasing ligand clearance. In particular, esRAGE constitutes ~5% of circulating RAGE in humans.

Higher circulating levels of esRAGE are associated with improved health outcomes and longevity while lower esRAGE is associated with many disease states including but not limited to atherosclerosis, diabetes, the metabolic syndrome, cardiovascular mortality, anaemia, autism and various tumorigenic states. Treatment of diabetic mice with recombinant esRAGE reduces atherosclerosis, vascular inflammation, renal and retinal damage.

Aberrant splicing of RAGE (and therefore dysfunctional RAGE signalling) has been reported in diabetes, some cancers and Alzheimer's disease.

The skipping of exon 10 in esRAGE-type splicing is ascribed to the limitation of intron length in higher eukaryotes. Roughly 45 nucleotides must separate the 5′ splice site and branch point, and the minimum distance between the branch point and 3′ splice site appears to be approximately 18 nucleotides, respectively. Therefore introns shorter than 70 nucleotides are extremely rare in mammals and cannot be spliced out efficiently. When the esRAGE 5′ splice site in intron 9 is selected, the distance between this site and the 3′ splice site that borders exon 10 is 46 nucleotides, which is considerably shorter than the lower limit of the intron length. Therefore, the use of the downstream, esRAGE 5′ splice site of intron 9 and the inclusion of exon 10 would be mutually exclusive. Among the known splice variants analyzed, all variants that used the downstream esRAGE 5′ splice site in intron 9 skipped exon 10; in contrast, all variants that used the upstream RAGE 5′ splice site in intron 9 included exon 10. Thus, the available evidence indicates that the selection of either one of the two alternative 5′ splice sites in intron 9 couples with inclusion or exclusion of exon 10. The means of regulation of this splicing or an external means to modulate has been previously unknown.

### Antisense Oligonucleotides (AONs)

Antisense oligonucleotide (AON) are short, synthetic, antisense, modified strands of DNA or RNA that can selectively hybridise to pre-RNA/mRNA through Watson-Crick base pairing and selectively modulate the function of the target RNA.

The terms “AON” and “ASO” are both abbreviations of the term “antisense oligonucleotide” and are used interchangeably herein.

When AONs are used to modulate alternative splicing of mRNA, they are often referred to as splice-switching oligonucleotides (SSO). In the present invention, the terms AON and SSO may be used interchangeably. SSOs base-pair with a pre-mRNA and disrupt the normal splicing repertoire of the transcript by blocking the RNA-RNA base-pairing or protein-RNA binding interactions that occur between components of the splicing machinery and the pre-mRNA. SSOs can induce “skipping” of selected exons and/or retention of intronic sequences to modulate the product of translation. This can be achieved by targeting splice sites directly or by targeting cis-acting sequences involved in enhancing or silencing splicing by modulating binding of specific proteins or altering secondary structure of the pre mRNA.

Therapeutic SSOs may be used for the treatment of genetic disorders, to skip faulty or misaligned sections allowing for the generation of internally deleted, but now functional protein as a therapy.

The AONs for use in the methods of the present invention selectively manipulate the alternative splicing pattern of RAGE pre-mRNA, resulting in the generation of either natural RAGE mRNA splicoforms that are either non-functional or that act as a decoy receptor to antagonise ligand dependent activation and ligand-independent transactivation of full length RAGE.

Notably, there are no common RAGE polymorphisms at these splice sites. The RAGE sequence is highly conserved. Therefore personalisation or individualised sequence modification is not required, unlike the management of genetic disorders with exon skipping technologies.

According to any aspect of the methods of the invention, the AONs as described herein are capable of binding to a selected target on a RAGE gene transcript to modulate pre-mRNA splicing in a RAGE gene transcript or part thereof. Broadly, the AON may be an isolated or purified AON. By “isolated” is meant material that is substantially or essentially free from components that normally accompany it in its native state. For example, an “isolated polynucleotide” or “isolated oligonucleotide,” as used herein, may refer to a polynucleotide that has been purified or removed from the sequences that flank it in a naturally-occurring state, e.g., a DNA fragment that is removed from the sequences that are adjacent to the fragment in the genome. The term “isolating” as it relates to cells, refers to the purification of cells (e.g., fibroblasts, lymphoblasts) from a source subject (e.g., a subject with a polynucleotide repeat disease). In the context of DNA, RNA or protein, “isolating” refers to the recovery of the DNA, mRNA or protein from a source, e.g., cells.

According to any aspect of the methods of the invention, the AONs as described herein promote splicing in the RAGE pre-mRNA resulting in the inclusion of exon 9b and/or exclusion (eg skipping) of exon 10, resulting in a premature stop and the complete loss of the trans-membrane and cytoplasmic domains. Therefore, the AONs as described herein may increase the level of RAGE_v1, RAGE_v6, RAGE_v8, RAGE_v9, RAGE_v10, RAGE_v15, RAGE_v18, and RAGE_v19 mRNA, preferably an increase in the level of RAGE_v1, RAGE_v6, RAGE_v8, RAGE_v9, RAGE_v10, RAGE_v15, RAGE_v18, and RAGE_v19 mRNA in a tissue or sample. For example, an AON as described herein may increase RAGE_v1 mRNA levels in a tissue or sample by promoting splicing in the RAGE pre-mRNA that results in the skipping of exon 10 and retention of intron 9 (exon 9b). In another example, an AON as described herein may increase RAGE_v10 mRNA levels in a tissue or sample by promoting splicing in the RAGE pre-mRNA that results in the skipping of exon 10 and exon 11.

An AON can be said to be “directed to” or “targeted against” a target sequence with which it hybridizes. In certain embodiments, the target sequence includes a region including a 3′ or 5′ splice site of a pre-processed mRNA, a branch point, or other sequences involved in the regulation of splicing, including splice enhancers and splice silencers and sites determining the secondary structure of RNA that influence splicing. The target sequence may be within an exon or within an intron or spanning an intron/exon junction.

In any aspect, the antisense oligonucleotide comprises, consists essentially of or consists of a nucleotide sequence at least 80%, 81%, 82%, 83%, 84%, 85%, 86%, 87%, 88%, 89%, 90%, 91%, 92%, 93%, 94%, 95%, 96%, 97%, 98%, 99% or 100% complementary to a target region of exon 10 of RAGE pre-mRNA over the entirety of the antisense oligonucleotide. Preferably, the 5′-most nucleotide of the AON is nucleotide position 88 or 114 of exon 10 or is between nucleotide positions 88 to 114 of exon 10. Preferably, the 5′-most nucleotide of the target region is nucleotide position 88 or 114 of exon 10 or is between nucleotide positions 88 to 114 of exon 10. Typically, the nucleotide position may be identical or equal to SEQ ID NO: 32, where the first nucleotide is position 1.

| | |
|---|---|
| Human RAGE | GCTCTGTGGGAGGATCAGGGCTGGGAACTCTAGCCCTGGCCCTGGGGATCC |
| Exon 10 | TGGGAGGCCTGGGGACAGCCGCCCTGCTCATTGGGGTCATCTTGTGGCAAA |
| (SEQ ID NO: 32) | GGCGGCAACGCCGAGGAGAGGAGAG |

In certain embodiments, the AON has sufficient sequence complementarity to a target RNA (i.e., the RNA for which splice site selection is modulated) to block a region of a target RNA (e.g., pre-mRNA) in an effective manner. In exemplary embodiments, such blocking of RAGE pre-mRNA serves to modulate splicing, either by masking a binding site for a splicosomal protein that would otherwise modulate splicing and/or by altering the structure of the targeted RNA. In some embodiments, the target RNA is target pre-mRNA (e.g., RAGE gene pre-mRNA).

An AON having a sufficient sequence complementarity to a target RNA sequence to modulate splicing of the target RNA means that the AON has a sequence sufficient to trigger the masking of a binding site for a native protein that would otherwise modulate splicing and/or alters the three-dimensional structure of the targeted RNA.

Selected AONs can be made shorter, e.g., about 12 bases, or longer, e.g., about 50 bases, and include a small number of mismatches, as long as the sequence is sufficiently complementary to effect splice modulation upon hybridization to the target sequence, and optionally forms with the RNA a heteroduplex having a Tm of 45° C. or greater.

Preferably, the AON is selected from the group comprising SEQ ID NOS: 1-31 and/or the sequences set forth in any of Tables 1a-1d. More preferably, the AON is SEQ ID NO: 11, 18, 19, or 20.

In certain embodiments, the degree of complementarity between the target sequence and AON is sufficient to form a stable duplex. The region of complementarity of the AONs with the target RNA sequence may be as short as 8-11 bases, but can be 12-15 bases or more, e.g., 10-50 bases, 10-40 bases, 12-30 bases, 12-25 bases, 15-25 bases, 12-20 bases, or 15-20 bases, including all integers in between these ranges. An AON of about 16-17 bases is generally long enough to have a unique complementary sequence. In certain embodiments, a minimum length of complementary bases may be required to achieve the requisite binding Tm, as discussed herein.

In certain embodiments, oligonucleotides as long as 50 bases may be suitable, where at least a minimum number of bases, e.g., 10-12 bases, are complementary to the target sequence. In general, however, facilitated or active uptake in cells is optimized at oligonucleotide lengths of less than about 30 bases. For phosphorodiamidate morpholino oligomer (PMO) AONs described further herein, an optimum balance of binding stability and uptake generally occurs at lengths of 18-25 bases. Included are AONs (e.g., PMOs, PMO-X, PNAs, LNAs, TINA, 2′-OMe) that consist of about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 bases.

In certain embodiments, AONs may be 100% complementary to the target sequence, or may include mismatches, e.g., to accommodate variants, as long as a heteroduplex formed between the oligonucleotide and target sequence is sufficiently stable to withstand the action of cellular nucleases and other modes of degradation which may occur in vivo. Hence, certain oligonucleotides may have about or at least about 70% sequence complementarity, e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence complementarity, between the oligonucleotide and the target sequence.

Mismatches, if present, are typically less destabilizing toward the end regions of the hybrid duplex than in the middle. The number of mismatches allowed will depend on the length of the oligonucleotide, the percentage of G:C base pairs in the duplex, and the position of the mismatch(es) in the duplex, according to well understood principles of duplex stability. Although such an AON is not necessarily 100% complementary to the target sequence, it is effective to stably and specifically bind to the target sequence, such that splicing of the target pre-RNA is modulated.

The stability of the duplex formed between an AON and a target sequence is a function of the binding Tm and the susceptibility of the duplex to cellular enzymatic cleavage. The Tm of an oligonucleotide with respect to complementary-sequence RNA may be measured by conventional methods, such as those described by Hames et al., Nucleic Acid Hybridization, IRL Press, 1985, pp. 107-108 or as described in Miyada C. G. and Wallace R. B., 1987, Oligonucleotide Hybridization Techniques, Methods Enzymol. Vol. 154 pp. 94-107. In certain embodiments, AONs may have a binding Tm, with respect to a complementary-sequence RNA, of greater than body temperature and preferably greater than about 45° C. or 50° C. Tm's in the range 60-80° C. or greater are also included.

Additional examples of variants include AONs having about or at least about 70% sequence identity or homology, e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity or homology, over the entire length of any of SEQ ID NOS: 1-31 and/or the sequences set forth in any of Tables 1a-1d. More preferably, the AON is SEQ ID NO: 11, 18, 19, or 20.

The modification of pre-mRNA splicing preferably induces “skipping”, or the removal of one or more exons or retention of introns of the mRNA. The resultant protein is preferably of a shorter length when compared to the parent full-length RAGE protein due to either internal truncation or premature termination. Preferably, the resultant protein has a C-terminal truncation. These truncated RAGE proteins may be termed splicoforms of the full length RAGE protein.

The remaining exons of the mRNA generated may be in-frame and produce a shorter protein with a sequence that is similar to that of the parent full length protein, except that it has an internal truncation in a region between the original 3′ and 5′ ends. In another possibility, the exon skipping may induce a frame shift that results in a protein wherein the first part of the protein is substantially identical to the parent full length protein, but wherein the second part of the protein has a different sequence (e.g. a nonsense sequence) due to a frame-shift. Alternatively, the exon skipping may induce the production of a prematurely terminated protein due to a disruption of the reading frame and presence of a premature termination of translation. The prematurely terminated protein may be the result of mRNA that is prematurely terminated (e.g. skipping of exons 10 and/or 11) or may be the result of a run on into an intron (e.g. RAGE 9b) or missense skip which provides an mRNA that contains the exon 10 and/or 11 mRNA, but which does not provide expression of the protein encoded by these exons.

Skipping individual exons of exons 1 to 9 will preferably disrupt the reading frame of the RAGE transcript. This will lead to increased degradation of RNA through nonsense mediated decay.

Skipping individual exons of exons 1 to 11 will preferably keep the reading frame intact. This will preferably lead to translation into an internally truncated protein. The truncated protein or RAGE mRNA splicoform may have a completely ablated function, may have a reduced function or act as a decoy receptor.

Preferably, these truncated, nonsense or prematurely terminated proteins are lacking one or more functional domains involved the induction of intracellular signalling pathways by RAGE ligands or non-ligand-dependent transactivation of RAGE by collocated GPCRs. For example, Exon 10 encodes a transmembrane domain and removing this exon may generate a soluble RAGE protein, which could potentially act as a soluble decoy or competitive antagonist of ligand induced signalling via RAGE. Truncated, nonsense or prematurely terminated proteins may further lack an attachment or binding site for other factors, removal of which may lead to a reduction in interaction of the RAGE protein with relevant signalling pathways.

Alternatively, the removal of one or more exons may lead to misfolding of the RAGE protein and a reduction in the ability of the protein to be successfully transported through the membrane.

The presence of internally truncated proteins (i.e. proteins lacking the amino acids encoded by one or more exons) is preferable. If the RAGE protein is inhibited, there may be problems with elevation of RAGE transcription as the body tries to compensate for the reduction in the total amount of RAGE protein. In contrast, the presence of an internally truncated protein (preferably lacking one or more of the features of the complete RAGE protein), should be sufficient to prevent elevated transcription, but still provide a therapeutic advantage due to a reduction in the total amount of functional RAGE protein.

The AON induced exon skipping as described herein need not completely or even substantially ablate the function of the RAGE protein. Preferably, the modulation of alternative splicing via the exon skipping process results in a reduced or compromised functionality of the RAGE protein.

The different isoforms of RAGE produced using different skipping strategies could result in proteins with ablated or reduced signalling activity that could preferably be used to treat or prevent different diseases associated with RAGE activity, such as neurodegenerative diseases, cancer, lung disorders, or inflammatory diseases. Alternative splicing strategies may form truncated proteins or proteins with reduced functions that can be preferably used as treatments for specific aspects, forms or progression of the diseases associated with RAGE expression and activity.

The skipping process using AONs may exclude (skip) an individual exon, or may result in skipping two or more exons at once.

The skipping process using AONs may include retention of intronic sequences with or without directly skipping one or more exons.

The AONs for use in the methods of the present invention may be a combination of two or more AONs capable of binding to a selected target to induce exon exclusion in a RAGE gene transcript. The combination may be a cocktail of two or more AONs and/or a construct comprising two or more or two or more AONs joined together.

TABLE 1a
Sequence of AONs for modulation of alternative splicing in human RAGE
Exon 9

| SEQ ID NO | AON# | Name | Sequence (5′ to 3′) |
|---|---|---|---|
| 1 | 3668 | H-AGERex9A(−10+15) | CCUCCUCGCCUGGUUCUGGAAGACA |
| 2 | 3669 | H-AGERex9A(+02+21) | UUGGCCCCUCCUCGCCUGGU |
| 3 | 3670 | H-AGERex9A(+08+27) | CUGCAGUUGGCCCCUCCUCG |
| 4 | 3671 | H-AGERex9D(+07−13) | UCAAACCCCUCACCUGCAGU |
| 5 | 4103 | H-AGERex9A(+3+27) | CCUGCAGUUGGCCCCUCCUCGCCUG |
| 6 | 4104 | H-AGERex9A(+10+27) | CCCCUCACCUGCAGUUGGCCCCUCC |
| 7 | 4105 | H-AGERex9A(+8+25) | GCAGUUGGCCCCUCCUC |
| 8 | 4106 | H-AGERex9D(+22−3) | CACCUGCAG UUG GCCCCUCCUCGCC |

TABLE 1b
Sequence of AONs for modulation of alternative splicing in human RAGE
Exon 10

| SEQ ID NO | AON# | Name | Sequence (5′ to 3′) |
|---|---|---|---|
| 9 | 3777 | AGER_H10A(−10+15) | AUCCUCCCACAGAGCCUGUACGGAG |
| 10 | 3778 | AGER_H10A(−5+20) | CCCUGAUCCUCCCACAGAGCCUGUA |
| 11 | 3779 | AGER_H10A(+90+114) | GGCGUUGCCGCCUUUGCCACAAGAU |
| 12 | 3780 | AGER_H10D(+20−5) | CUCACCUCUCCUCUCCUCGGCGUUG |
| 13 | 3781 | AGER_H10D(+15−10) | CUCCACUCACCUCUCCUCUCCUCGG |
| 14 | 82 | AGER_H10A (+60+79) | AGCAGGGCGGCUGUCCCCAG |
| 15 | 83 | AGER_H10A (+75+99) | GCCACAAGAUGACCCCAAUGAGCAG |
| 16 | 84 | AGER_A10 (+80+104) | CCUUUGCCACAAGAUGACCCCAAUG |
| 17 | 85 | AGER_A10 (+85+109) | UGCCGCCUUUGCCACAAGAUGACCC |
| 18 | 87 | AGER_H10(+95+119) | UCCUCGGCGUUGCCGCCUUUGCCAC |
| 19 | 90 | AGER_H10A(+90+102) | UUUGCCACAAGAU |
| 20 | 93 | AGER_H10A(+88+107) | CCGCCUUUGCCACAAGAUGA |

TABLE 1c
Sequence of AONs for modulation of alternative splicing in human RAGE
Intron 9

| SEQ ID NO | AON# | Name | Sequence (5′ to 3′) |
|---|---|---|---|
| 21 | 88 | AGER_H10A(−75−51) | UUUCUUGUUGACCAUCCCCCCAGUC |
| 22 | 89 | AGER_H10A(−86−62) | CCAUCCCCCCAGUCACAUGUGUUGG |

TABLE 1d
Sequence of AONs for modulation of alternative splicing in murine RAGE

| SEQ ID NO | AON# | Name | Sequence (5′ to 3′) |
|---|---|---|---|
| 23 | 3672 | M-AgerEx9A(−10+15) | CCUCCUCGCCUGGUUCUGGAAGACA |
| 24 | 3673 | M-AgerEx9A(+02+21) | CUGGCCCCUCAUCGCCGGUU |
| 25 | 3674 | M-AgerEx9A(+08+27) | CUUCAGCUGGCCCCUCAUCG |
| 26 | 3675 | M-AgerEx9D(+07−13) | UCCAGUCCCUCACCUUCAGC |
| 27 | m3779 | M-AgerEx10A(+90+114) | UGGGUUGUCGUUUUCGCCACAGGAU |
| | (m79) | | |
| 28 | m97 | M-AgerEx10A(+48,+70) | ACUACUCCCAGGCCUCCCAGGAU |
| 29 | m98 | M-AgerEx10A(+56+79) | GCAGGGCUACUACUCCCAGGCA |
| 30 | m101 | M-AgerEx10D(−15+10) | UCUCACUCACCUCUCCUCACGCCUG |
| 31 | m102 | M-AgerEx10A(−5+20) | CCCAGACUCACCCACAGAGCCUGUA |
| 34 | ASO2 | M-AgerEx10A(+90+107) | TCGTTTTCGCCACAGGAT |

More specifically, the AON for use in the methods of the present invention may be selected from those set forth in any of Tables 1a-1d. The sequences are preferably selected from the group consisting of any one or more of any one or more of SEQ ID NOs: 1-31, and combinations or cocktails thereof. More preferably, the AON is SEQ ID NO: 11, 18, 19, or 20. The combination of AONs is preferably a combination of SEQ ID NO: 11 and 10, or SEQ ID NO: 11 and 13. This includes sequences which can hybridise to such sequences under stringent hybridisation conditions, sequences complementary thereto, sequences containing modified bases, modified backbones, and functional truncations or extensions thereof which possess or modulate pre-mRNA processing activity in a RAGE gene transcript.

The oligomer and the DNA, cDNA or RNA are complementary to each other when a sufficient number of corresponding positions in each molecule are occupied by nucleotides which can hydrogen bond with each other. Thus, “specifically hybridisable” and “complementary” are terms which are used to indicate a sufficient degree of complementarity or pairing such that stable and specific binding occurs between the oligomer and the DNA, cDNA or RNA target. It is understood in the art that the sequence of an AON need not be 100% complementary to that of its target sequence to be specifically hybridisable. An AON is specifically hybridisable when binding of the compound to the target DNA or RNA molecule interferes with the normal function of the target DNA or RNA product, and there is a sufficient degree of complementarity to avoid non-specific binding of the AON to non-target sequences under conditions in which specific binding is desired, i.e., under physiological conditions in the case of in vivo assays or therapeutic treatment, and in the case of in vitro assays, under conditions in which the assays are performed.

Selective hybridisation may be under low, moderate or high stringency conditions, but is preferably under high stringency. Those skilled in the art will recognise that the stringency of hybridisation will be affected by such conditions as salt concentration, temperature, or organic solvents, in addition to the base composition, length of the complementary strands and the number of nucleotide base mismatches between the hybridising nucleic acids. Stringent temperature conditions will generally include temperatures in excess of 30° C., typically in excess of 37° C., and preferably in excess of 45° C., preferably at least 50° C., and typically 60° C.-80° C. or higher. Stringent salt conditions will ordinarily be less than 1000 mM, typically less than 500 mM, and preferably less than 200 mM. However, the combination of parameters is much more important than the measure of any single parameter. An example of stringent hybridisation conditions is 65° C. and 0.1×SSC (1×SSC=0.15 M NaCl, 0.015 M sodium citrate pH 7.0). Thus, the AONs for use in the methods of the present invention may include oligomers that selectively hybridise to the sequences provided in any of Tables 1a-1d, or SEQ ID NOs: 1-31. More preferably, the AON is SEQ ID NO: 11, 18, 19, or 20.

It will be appreciated that the codon arrangements at the end of exons in structural proteins may not always break at the end of a codon, consequently there may be a need to delete more than one exon from the pre-mRNA to ensure in-frame reading of the mRNA. In such circumstances, a plurality of AONs may need to be selected by the method of the invention wherein each is directed to a different region responsible for inducing inclusion of the desired exon and/or intron. At a given ionic strength and pH, the Tm is the temperature at which 50% of a target sequence hybridizes to a complementary polynucleotide. Such hybridization may occur with “near” or “substantial” complementarity of the AON to the target sequence, as well as with exact complementarity.

Typically, selective hybridisation will occur when there is at least about 55% identity over a stretch of at least about 14 nucleotides, preferably at least about 65%, more preferably at least about 75% and most preferably at least about 90%, 95%, 98% or 99% identity with the nucleotides of the AON. The length of homology comparison, as described, may be over longer stretches and in certain embodiments will often be over a stretch of at least about nine nucleotides, usually at least about 12 nucleotides, more usually at least about 20, often at least about 21, 22, 23 or 24 nucleotides, at least about 25, 26, 27 or 28 nucleotides, at least about 29, 30, 31 or 32 nucleotides, at least about 36 or more nucleotides.

Thus, the AON for use in the methods of the present invention sequences preferably have at least 75%, more preferably at least 85%, more preferably at least 86, 87, 88, 89 or 90% homology to the sequences shown in the sequence listings herein. More preferably there is at least 91, 92, 93 94, or 95%, more preferably at least 96, 97, 98% or 99%, homology. Generally, the shorter the length of the AON, the greater the homology required to obtain selective hybridisation. Consequently, where an AON consists of less than about 30 nucleotides, it is preferred that the percentage identity is greater than 75%, preferably greater than 85, 86, 87, 88, 89, 90, 91, 92, 93, 94, 95%, 96, 97, 98% or 99% compared with the AONs set out in the sequence listings herein. Nucleotide homology comparisons may be conducted by sequence comparison programs such as the GCG Wisconsin Bestfit program or GAP (Deveraux et al., 1984, Nucleic Acids Research 12, 387-395). In this way sequences of a similar or substantially different length to those cited herein could be compared by insertion of gaps into the alignment, such gaps being determined, for example, by the comparison algorithm used by GAP.

The AONs for use in the methods of the present invention may have regions of reduced homology, and regions of exact homology with the target sequence. It is not necessary for an oligomer to have exact homology for its entire length. For example, the oligomer may have continuous stretches of at least 4 or 5 bases that are identical to the target sequence, preferably continuous stretches of at least 6 or 7 bases that are identical to the target sequence, more preferably continuous stretches of at least 8 or 9 bases that are identical to the target sequence. The oligomer may have stretches of at least 10, 11, 12, 13, 14, 15, 16, 17, 18, 19, 20, 21, 22, 23, 24, 25 or 26 bases that are identical to the target sequence. The remaining stretches of oligomer sequence may be intermittently identical with the target sequence; for example, the remaining sequence may have an identical base, followed by a non-identical base, followed by an identical base. Alternatively (or as well) the oligomer sequence may have several stretches of identical sequence (for example 3, 4, 5 or 6 bases) interspersed with stretches of less than perfect homology. Such sequence mismatches will preferably have no or very little loss of splice switching activity.

The term “modulate” or “modulates” includes to “increase” or “decrease” one or more quantifiable parameters, optionally by a defined and/or statistically significant amount. The terms “increase” or “increasing,” “enhance” or “enhancing,” or “stimulate” or “stimulating” refer generally to the ability of one or AONs or compositions to produce or cause a greater physiological response (i.e., downstream effects) in a cell or a subject relative to the response caused by either no AON or a control compound. The terms “decreasing” or “decrease” refer generally to the ability of one or AONs or compositions to produce or cause a reduced physiological response (i.e., downstream effects) in a cell or a subject relative to the response caused by either no AON or a control compound.

Relevant physiological or cellular responses (in vivo or in vitro) will be apparent to persons skilled in the art, and may include increases in the exclusion of specific exons in a RAGE-coding pre-mRNA, decreases in the amount of RAGE-coding pre-mRNA or decreases in the expression of functional RAGE protein in a cell, tissue, or subject in need thereof. An “increased” or “enhanced” amount is typically a statistically significant amount, and may include an increase that is 1.1, 1.2, 2, 3, 4, 5, 6, 7, 8, 9, 10, 15, 20, 30, 40, 50 or more times (e.g., 500, 1000 times) (including all integers and decimal points in between and above 1, e.g., 1.5, 1.6, 1.7, 1.8) the amount produced by no AON (the absence of an agent) or a control compound. The term “reduce” or “inhibit” may relate generally to the ability of one or more AONs or compositions to “decrease” a relevant physiological or cellular response, such as a symptom of a disease or condition described herein, as measured according to routine techniques in the diagnostic art. Relevant physiological or cellular responses (in vivo or in vitro) will be apparent to persons skilled in the art, and may include reductions in the symptoms or pathology of a disease such as cancer, neurodegenerative diseases, lung disorders, and other inflammatory diseases. A “decrease” in a response may be statistically significant as compared to the response produced by no AON or a control composition, and may include a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% decrease, including all integers in between.

The length of an AON may vary, as long as it is capable of binding selectively to the intended location within the pre-mRNA molecule. The length of such sequences can be determined in accordance with selection procedures described herein. Generally, the AON will be from about 10 nucleotides in length, up to about 50 nucleotides in length. It will be appreciated, however, that any length of nucleotides within this range may be used in the method. Preferably, the length of the AON is between 10 and 40, 10 and 35, 15 to 30 nucleotides in length or 20 to 30 nucleotides in length, most preferably about 25 to 30 nucleotides in length. For example, the oligomer may be 20, 21, 22, 23, 24, 25, 26, 27, 28, 29 or 30 nucleotides in length.

As used herein, an “AON” refers to a linear sequence of nucleotides, or nucleotide analogs, that allows the nucleobase to hybridize to a target sequence in an RNA by Watson-Crick base pairing, to form an oligonucleotide: RNA heteroduplex within the target sequence. The terms “AON”, “AON”, “oligomer” and “antisense compound” may be used interchangeably to refer to an oligonucleotide. The cyclic subunits may be based on ribose or another pentose sugar or, in certain embodiments, a morpholino group (see description of morpholino oligonucleotides below). Also contemplated are peptide nucleic acids (PNAs), locked nucleic acids (LNAs), and 2′-O-Methyl oligonucleotides, 2′-O-Methoxyethyl oligonucleotides, among other antisense agents known in the art.

Included are non-naturally-occurring AONs, or “oligonucleotide analogs”, including AONs or oligonucleotides having (i) a modified backbone structure, e.g., a backbone other than the standard phosphodiester linkage found in naturally-occurring oligo- and polynucleotides, and/or (ii) modified sugar moieties, e.g., morpholino moieties rather than ribose or deoxyribose moieties. Oligonucleotide analogs support bases capable of hydrogen bonding by Watson-Crick base pairing to standard polynucleotide bases, where the analog backbone presents the bases in a manner to permit such hydrogen bonding in a sequence-specific fashion between the oligonucleotide analog molecule and bases in a standard polynucleotide (e.g., single-stranded RNA or single-stranded DNA). Preferred analogs are those having a substantially uncharged, phosphorus containing backbone.

One method for producing AONs is the methylation of the 2′ hydroxyribose position and the incorporation of a phosphorothioate backbone produces molecules that superficially resemble RNA but that are much more resistant to nuclease degradation, although persons skilled in the art of the methods of the invention will be aware of other forms of suitable backbones that may be useable in the objectives of the methods of the invention.

Increased splice-switching may also be achieved with alternative oligonucleotide chemistry. For example, the AON may be chosen from the list comprising: phosphoramidate or phosphorodiamidate morpholino oligomer (PMO); PMO-X; PPMO; peptide nucleic acid (PNA); a locked nucleic acid (LNA) and derivatives including alpha-L-LNA, 2′-amino LNA, 4′-methyl LNA and 4′-O-methyl LNA; ethylene bridged nucleic acids (ENA) and their derivatives; phosphorothioate oligomer; tricyclo-DNA oligomer (tcDNA); tricyclophosphorothioate oligomer; 2′O-Methyl-modified oligomer (2′-OMe); 2′-O-methoxy ethyl(2′-MOE); 2′-fluoro, 2′-fluroarabino (FANA); unlocked nucleic acid (UNA); thermostable twisted intercalating nucleic acid (TINA), hexitol nucleic acid (HNA); cyclohexenyl nucleic acid (CeNA); 2′-amino (2′—NH2); 2′-O-ethyleneamine or any combination of the foregoing as mixmers or as gapmers. To further improve the delivery efficacy, the above mentioned modified nucleotides are often conjugated with fatty acids/lipid/cholesterol/amino acids/carbohydrates/polysaccharides/nanoparticles etc. to the sugar or nucleobase moieties. These conjugated nucleotide derivatives can also be used to construct exon skipping AONs. Antisense oligonucleotide-induced splice modification of the human RAGE gene transcripts have generally used either oligoribonucleotides, PNAs, 2OMe or MOE modified bases on a phosphorothioate backbone. When alternative chemistries are used to generate the AONs for use in the methods of the invention, the uracil (U) of the sequences provided herein may be replaced by a thymine (T).

Included within the AONs for use in the methods of the present invention are non-naturally-occurring oligomers, or “oligonucleotide analogues,” including oligomers having (i) a modified backbone structure, e.g., a backbone other than the standard phosphodiester linkage found in naturally-occurring oligo- and polynucleotides, and/or (ii) modified sugar moieties, e.g., morpholino moieties rather than ribose or deoxyribose moieties. Oligomer analogues support bases capable of hydrogen bonding by Watson-Crick base pairing to standard polynucleotide bases, where the analogue backbone presents the bases in a manner to permit such hydrogen bonding in a sequence-specific fashion between the oligomer analogue molecule and bases in a standard polynucleotide (e.g., single-stranded RNA or single-stranded DNA). Preferred analogues are those having a substantially uncharged, phosphorus containing backbone.

Antisense oligonucleotides that do not activate RNase H can be made in accordance with known techniques (see, e.g., U.S. Pat. No. 5,149,797). Such AONs, which may be deoxyribonucleotide or ribonucleotide sequences, simply contain any structural modification which sterically hinders or prevents binding of RNase H to a duplex molecule containing the oligomer as one member thereof, which structural modification does not substantially hinder or disrupt duplex formation. Because the portions of the oligomer involved in duplex formation are substantially different from those portions involved in RNase H binding thereto, numerous AONs that do not activate RNase H are available. For example, such AONs may be oligomers wherein at least one, or all, of the inter-nucleotide bridging phosphate residues are modified phosphates, such as methyl phosphonates, methyl phosphorothioates, phosphoromorpholidates, phosphoropiperazidates boranophosphates, amide linkages and phosphoramidates. For example, every other one of the internucleotide bridging phosphate residues may be modified as described. In another non-limiting example, such AONs are molecules wherein at least one, or all, of the nucleotides contain a 2′ lower alkyl moiety (such as, for example, C₁-C₄, linear or branched, saturated or unsaturated alkyl, such as methyl, ethyl, ethenyl, propyl, 1-propenyl, 2-propenyl, and isopropyl). For example, every other one of the nucleotides may be modified as described.

Specific examples of preferred AONs for use in the methods of the present invention include oligomers containing modified backbones or non-natural inter-nucleoside linkages. As defined in this specification, oligomers having modified backbones include those that retain a phosphorus atom in the backbone and those that do not have a phosphorus atom in the backbone. For the purposes of this specification, and as sometimes referenced in the art, modified oligomers that do not have a phosphorus atom in their inter-nucleoside backbone can also be considered to be AONs.

In other preferred oligomer mimetics, both the sugar and the inter-nucleoside linkage, i.e., the backbone, of the nucleotide units are replaced with novel groups. The base units are maintained for hybridization with an appropriate nucleic acid target compound. One such oligomeric compound, an oligomer mimetic that has been shown to have excellent hybridization properties, is referred to as a peptide nucleic acid (PNA). In PNA compounds, the sugar-backbone of an oligomer is replaced with an amide containing backbone, in particular an aminoethylglycine backbone. The nucleo-bases are retained and are bound directly or indirectly to aza nitrogen atoms of the amide portion of the backbone.

Another preferred chemistry is the phosphorodiamidate morpholino oligomer (PMO) oligomeric compounds, which are not degraded by any known nuclease or protease. These compounds are uncharged, do not activate RNase H activity when bound to a RNA strand and have been shown to exert sustained splice modulation after in vivo administration (Summerton and Weller, Antisense Nucleic Acid Drug Development, 7, 187-197).

Modified oligomers may also contain one or more substituted sugar moieties. Oligomers may also include nucleobase (often referred to in the art simply as “base”) modifications or substitutions. Certain nucleobases are particularly useful for increasing the binding affinity of the oligomeric compounds as described herein. These include 5-substituted pyrimidines, 6-azapyrimidines, and N-2, N-6 and O-6 substituted purines, including 2-aminopropyladenine, 5-propynyluracil, 5-propynylcytosine and 5-methylcytosine substitutions have been shown to increase nucleic acid duplex stability by 0.6-1.2° C., even more particularly when combined with 2′-O-methoxyethyl sugar modifications.

The activity of AONs and variants thereof can be assayed according to routine techniques in the art. For example, splice forms and expression levels of surveyed RNAs and proteins may be assessed by any of a wide variety of well-known methods for detecting splice forms and/or expression of a transcribed nucleic acid or protein. Non-limiting examples of such methods include RT-PCR of spliced forms of RNA followed by size separation of PCR products, nucleic acid hybridization methods e.g., Northern blots and/or use of nucleic acid arrays; nucleic acid amplification methods; immunological methods for detection of proteins; protein purification methods; and protein function or activity assays.

RNA expression levels can be assessed by preparing mRNA/cDNA (i.e., a transcribed polynucleotide) from a cell, tissue or organism, and by hybridizing the mRNA/cDNA with a reference polynucleotide, which is a complement of the assayed nucleic acid, or a fragment thereof. cDNA can, optionally, be amplified using any of a variety of polymerase chain reaction or in vitro transcription methods prior to hybridization with the complementary polynucleotide; preferably, it is not amplified. Expression of one or more transcripts can also be detected using quantitative PCR to assess the level of expression of the transcript(s).

The AONs for use in the methods of the present invention may provide induced splice-switching of the RAGE gene transcript, clinically relevant oligomer chemistries and delivery systems to direct RAGE splice manipulation to therapeutic levels, promotion of the non-signalling decoy-receptor RAGE mRNA splicoform (e.g. RAGE_v1, RAGE_v6, RAGE_v8, RAGE_v9, RAGE_v10, RAGE_v15, RAGE_v18, and RAGE_v19 mRNA) can be achieved, and therefore may be used to treat a RAGE-related respiratory (pulmonary) disease or disorder.

The AONs for use in the methods of the present invention may be conveniently made through the well-known technique of solid phase synthesis. Equipment for such synthesis is sold by several vendors including, for example, Applied Biosystems (Foster City, Calif.). One method for synthesising oligomers on a modified solid support is described in U.S. Pat. No. 4,458,066.

Any other means for such synthesis known in the art may additionally or alternatively be employed. It is well known to use similar techniques to prepare oligomers such as the phosphorothioates and alkylated derivatives. In one such automated embodiment, diethyl-phosphoramidites are used as starting materials and may be synthesized as described by Beaucage, et al., (1981) Tetrahedron Letters, 22:1859-1862.

The AONs for use in the methods of the present invention are synthesised in vitro and do not include antisense compositions of biological origin, or genetic vector constructs designed to direct the in vivo synthesis of AONs.

### Administration, Dosage and Formulation

In any aspect, the methods of the present invention may be used to prevent or treat a disease associated with, or caused by, RAGE in a subject. Preferably, the disease associated with, or caused by, RAGE is a respiratory (pulmonary) disorder or disease and is selected from the group: Acute upper respiratory infections, rhinitis, nasopharyngitis, sinusitis, laryngitis, influenza and pneumonia, acute bronchitis, acute bronchiolitis, asthma, chronic obstructive pulmonary disease (COPD), bronchiectasis, emphysema, chronic lung diseases due to external agents, Acute Respiratory Distress Syndrome (ARDS), pulmonary eosinophilia, and pleuritic, lung trauma and recovery from lung injury, trauma and/or surgery.

The term ‘respiratory’ refers to the process by which oxygen is taken into the body and carbon dioxide is discharged, through the bodily system including the nose, throat, larynx, trachea, bronchi and lungs.

As used herein, the upper respiratory tract may include the following regions: nose and nasal passages, paranasal sinuses, the pharynx, and the portion of the larynx above the vocal folds (cords). Typically, the lower respiratory tract includes any one of more of the following regions: portion of the larynx below the vocal folds, trachea, bronchi and bronchioles. The lungs can be included in the lower respiratory tract and include the respiratory bronchioles, alveolar ducts, alveolar sacs, and alveoli.

The term ‘respiratory disease’ or ‘respiratory condition’ refers to any one of several ailments that involve inflammation and affect a component of the respiratory system including the upper (including the nasal cavity, pharynx and larynx) and lower respiratory tract (including trachea, bronchi and lungs).

A symptom of respiratory disease may include cough, excess sputum production, a sense of breathlessness or chest tightness with audible wheeze. Exercise capacity may be quite limited. In asthma the FEV1.0 (forced expiratory volume in one second) as a percentage of that predicted nomographically based on weight, height and age, may be decreased as may the peak expiratory flow rate in a forced expiration. In COPD the FEV1.0 as a ratio of the FVC is typically reduced to less than 0.7. The impact of each of these conditions may also be measured by days of lost work/school, disturbed sleep, requirement for bronchodilator drugs, requirement for glucocorticoids including oral glucocorticoids.

The existence of, improvement in, treatment of or prevention of a respiratory disease may be determined by any clinically or biochemically relevant method of the subject or a biopsy therefrom. For example, a parameter measured may be the presence or degree of lung function, signs and symptoms of obstruction; exercise tolerance; night time awakenings; days lost to school or work; bronchodilator usage; inhaled corticosteroid (ICS) dose; oral (glucocorticoid) GC usage; need for other medications; need for medical treatment; hospital admission.

The terms “treatment” or “treating” of a subject includes the application or administration of an AON as described herein with the purpose of delaying, slowing, stabilizing, curing, healing, alleviating, relieving, altering, remedying, less worsening, ameliorating, improving, or affecting the disease or condition, the symptom of the disease or condition, or the risk of (or susceptibility to) the disease or condition. The term “treating” refers to any indication of success in the treatment or amelioration of an injury, pathology or condition, including any objective or subjective parameter such as abatement; remission; lessening of the rate of worsening; lessening severity of the disease; stabilization, diminishing of symptoms or making the injury, pathology or condition more tolerable to the subject; slowing in the rate of degeneration or decline; making the final point of degeneration less debilitating; or improving a subject's physical or mental well-being.

The AONs as described herein may be are adapted to aid in the prophylactic or therapeutic treatment, prevention or amelioration of symptoms of a disease such as a RAGE expression related disease or pathology in a form suitable for delivery to a patient.

A positive response to therapy may also be prevention or attenuation of worsening of respiratory symptoms, e.g. asthma symptoms (exacerbation), following a respiratory virus infection. This could be assessed by comparison of the mean change in disease score from baseline to end of study period based on Juniper Asthma Control Questionnaire (ACQ-6), and could also assess lower respiratory symptom score (LRSS—symptoms of chest tightness, wheeze, shortness or breath and cough) daily following infection/onset of cold symptoms. Change from baseline lung function (peak expiratory flow PEF) could also be assessed and a positive response to therapy could be a significant attenuation in reduced PEF. For example, a placebo treated group would show a significant reduction in morning PEF of 15% at the peak of exacerbation whilst the treatment group would show a non-significant reduction in PEF less than 15% change from baseline.

Although the methods of the present invention finds application in humans, the methods of the invention is also useful for therapeutic veterinary purposes. The invention is useful for domestic or farm animals such as cattle, sheep, horses and poultry; for companion animals such as cats and dogs; and for zoo animals.

In certain embodiments, the AONs as described herein can be delivered by pulmonary or nasal routes (e.g., via nebulised saline incorporating the AONs). The highest endogenous expression of RAGE mRNA in the healthy human tissues is found in the lung and is accessible via the airways. Inhaled oligonucleotides are an emerging therapeutic modality for respiratory diseases. The airways are uniquely lined with pulmonary surfactants, which are primarily composed of zwitterionic lipids. These surfactant lipids possess cationic properties at the pH of the respiratory tract. When anionic oligonucleotides are inhaled, they tend to be adsorbed by the surfactants, resulting in reformulated particles that have been hypothesised to be efficiently taken up by bronchial and alveolar epithelial cells into the pulmonary cells. Of note, AONs have been shown to be able to withstand the nebulization process.

The AONs as described herein may be in compositions formulated for administration to the lower respiratory tract only. Limitation to the lower respiratory tract may be achieved by an amount, particularly volume and composition of form ie. particle size, physical form whether dry powder or solution droplet, of composition that would otherwise be administered to the upper respiratory tract. Alternatively, the AONs as described herein may be administered via a device that ensures retention in the lower respiratory tract only.

The AONs as described herein may be formulated for intranasal administration, including dry powder, sprays, mists, or aerosols. This may be particularly preferred for treatment of a respiratory infection.

Suitable formulations, wherein the carrier is a liquid, for administration, as for example, a nasal spray or as nasal drops, include aqueous or oily solutions of the active ingredient. Alternatively, the AONs as described herein may be provided as a dry powder and administered to the upper respiratory tract only as defined herein.

The selection of appropriate carriers depends upon the particular type of administration that is contemplated. For administration via the upper respiratory tract, e.g., the nasal mucosal surfaces, the compound can be formulated into a solution, e.g., water or isotonic saline, buffered or unbuffered, or as a suspension, for intranasal administration as drops or as a spray. Preferably, such solutions or suspensions are isotonic relative to nasal secretions and of about the same pH, ranging e.g., from about pH 4.0 to about pH 7.4 or, from pH 6.0 to pH 7.0. Buffers should be physiologically compatible and include, simply by way of example, phosphate buffers. For example, a representative nasal decongestant is described as being buffered to a pH of about 6.2 (Remington's, Id. at page 1445). Of course, the ordinary artisan can readily determine a suitable saline content and pH for an innocuous aqueous carrier for nasal and/or upper respiratory administration.

Other ingredients, such as art known preservatives, colorants, lubricating or viscous mineral or vegetable oils, perfumes, natural or synthetic plant extracts such as aromatic oils, and humectants and viscosity enhancers such as, e.g., glycerol, can also be included to provide additional viscosity, moisture retention and a pleasant texture and odour for the formulation. For nasal administration of solutions or suspensions according to the methods of the invention, various devices are available in the art for the generation of drops, droplets and sprays. For example, an AON as described herein can be administered into the nasal passages by means of a simple dropper (or pipet) that includes a glass, plastic or metal dispensing tube from which the contents are expelled drop by drop by means of air pressure provided by a manually powered pump, e.g., a flexible rubber bulb, attached to one end.

The delivery of a therapeutically useful amount of AONs may be achieved by methods previously published. For example, intracellular delivery of the AON may be via a composition comprising an admixture of the AON and an effective amount of a block copolymer. An example of this method is described in US patent application US20040248833. Other methods of delivery of AONs to the nucleus are described in Mann C J et al. (2001) Proc, Natl. Acad. Science, 98(1) 42-47, and in Gebski et al. (2003) Human Molecular Genetics, 12(15):1801-1811. A method for introducing a nucleic acid molecule into a cell by way of an expression vector either as naked DNA or complexed to lipid carriers, is described in U.S. Pat. No. 6,806,084.

The AONs for use in the methods or uses of the present invention is to be administered in an effective amount. The phrase ‘therapeutically effective amount’ or ‘effective amount’ generally refers to an amount of an AON, a pharmaceutically acceptable salt, polymorph or prodrug thereof as described herein that (i) treats the particular disease, condition, or disorder, (ii) attenuates, ameliorates, or eliminates one or more symptoms of the particular disease, condition, or disorder, or (iii) delays the onset of one or more symptoms of the particular disease, condition, or disorder described herein. Undesirable effects, e.g. side effects, are sometimes manifested along with the desired therapeutic effect; hence, a practitioner balances the potential benefits against the potential risks in determining what is an appropriate “effective amount”.

The exact amount required will vary from subject to subject, depending on the species, age and general condition of the subject, mode of administration and the like. Thus, it may not be possible to specify an exact “effective amount”. However, an appropriate “effective amount” in any individual case may be determined by one of ordinary skill in the art using only routine experimentation. In one aspect, the dose administered to a subject is any dose that reduces viral load.

The phrase “pharmaceutically acceptable” refers to molecular entities and compositions that are physiologically tolerable and do not typically produce an allergic or similarly untoward reaction, such as gastric upset and the like, when administered to a patient. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or saline solutions and aqueous dextrose and glycerol solutions are preferably employed as carriers, particularly for injectable solutions. Suitable pharmaceutical carriers are described in Martin, Remington's Pharmaceutical Sciences, 18th Ed., Mack Publishing Co., Easton, PA, (1990).

In any aspect, the method or use comprises administering an AON, as described herein, to the respiratory tract of the subject once every at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, or at least about 28 days after the first administration.

In any aspect, the method or use comprises administering and AON, as described herein, to the respiratory tract of the subject once every at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, or at least 28 days after the first administration.

In any aspect, the method or use comprises administering an AON, as described herein, to the respiratory tract of the subject once every 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, or 28 days after the first administration.

In any aspect, the method or use comprises administering an AON, as described herein, to the respiratory tract of the subject once every about 7 days to 28 days, about 8 days to 28 days, about 9 days to 28 days, about 10 days to 28 days, about 11 days to 28 days, about 12 days to 28 days, about 13 days to 28 days, about 14 days to about 28 days, about 15 days to about 28 days, about 16 days to about 28 days, about 17 days to about 28 days, about 18 days to about 28 days, about 19 days to about 28 days, about 20 days to about 28 days, about 21 days to about 28 days, about 22 days to about 28 days, about 23 days to about 28 days, about 24 days to about 28 days, about 25 days to about 28 days, or about 26 days to about 28 days after the first administration.

In any aspect, the method or use comprises administering an AON, as described herein, to the respiratory tract of the subject once every 7 days to 28 days, 8 days to 28 days, 9 days to 28 days, 10 days to 28 days, 11 days to 28 days, 12 days to 28 days, 13 days to 28 days, 14 days to 28 days, 15 days to 28 days, 16 days to 28 days, 17 days to 28 days, 18 days to 28 days, 19 days to 28 days, 20 days to 28 days, 21 days to 28 days, 22 days to 28 days, 23 days to 28 days, 24 days to 28 days, 25 days to 28 days, or 26 days to 28 days after the first administration.

In any aspect, the method or use comprises administering an AON, as described herein, to the respiratory tract of the subject once, no more than 28 days after the first administration.

In any aspect, the method or use comprises administering an AON, as described herein, to the respiratory tract of the subject in a first and second administration; in a first, second and third administration; or in a first, second, third and fourth administration. For example, a second administration is about 7 days after the first administration, a third administration is about 7 days after the second administration and a fourth administration is about 7 days after the third administration. In another example, a second administration is about 14 days after the first administration, a third administration is about 14 days after the second administration and a fourth administration is about 14 days after the third administration. In yet another example, a second administration is about 21 days after the first administration, a third administration is about 21 days after the second administration and a fourth administration is about 21 days after the third administration. In yet another example, a second administration is about 28 days after the first administration, a third administration is about 28 days after the second administration and a fourth administration is about 28 days after the third administration.

In any aspect, the method or use comprises administering an AON, as described herein, to the respiratory tract of the subject at a regular time interval.

In any aspect, the second or further administration of an AON, as described herein, is from about 7 days to 28 days, from about 8 days to 28 days, from about 9 days to 28 days, from about 10 days to 28 days, from about 11 days to 28 days, from about 12 days to 28 days, from about 13 days to 28 days, from about 14 days to about 28 days, about 15 days to about 28 days, about 16 days to about 28 days, about 17 days to about 28 days, about 18 days to about 28 days, about 19 days to about 28 days, about 20 days to about 28 days, about 21 days to about 28 days, about 22 days to about 28 days, about 23 days to about 28 days, about 24 days to about 28 days, about 25 days to about 28 days, or about 26 days to about 28 days after the first or previous administration.

In any aspect, the second or further administration of an AON, as described herein, is from 7 days to 28 days, from 8 days to 28 days, from 9 days to 28 days, from 10 days to 28 days, from 11 days to 28 days, from 12 days to 28 days, from 13 days to 28 days, from 14 days to 28 days, 15 days to 28 days, 16 days to 28 days, 17 days to 28 days, 18 days to 28 days, 19 days to 28 days, 20 days to 28 days, 21 days to 28 days, 22 days to 28 days, 23 days to 28 days, 24 days to 28 days, 25 days to 28 days, or 26 days to 28 days after the first or previous administration.

In any aspect, the second or further administration of an AON, as described herein, is no more than 28 days after the first or previous administration.

In any aspect or embodiment, each further administration of an AON as described herein is provided at a regular time interval as described herein. For example, a second administration is 7 days after the first administration, a third administration is 7 days after the second administration and a fourth administration is 7 days after the third administration. In another example, a second administration is 14 days after the first administration, a third administration is 14 days after the second administration and a fourth administration is 14 days after the third administration. In yet another example, a second administration is 21 days after the first administration, a third administration is 21 days after the second administration and a fourth administration is 21 days after the third administration. In yet another example, a second administration is 28 days after the first administration, a third administration is 28 days after the second administration and a fourth administration is 28 days after the third administration.

In any aspect, the further administrations of an AON as described herein is at least one administration, at least two administrations, or at least three administrations.

In any embodiment, an AON, as described herein, is administered in an amount and manner effective to result in a peak blood concentration of at least 200-400 nM AON.

In any embodiment the AON is administered in an amount of a human equivalent dose of those doses described in the preclinical studies in the Examples.

Treatment may be monitored, e.g., by general indicators of disease known in the art. The efficacy of an in vivo administered AONs for use in the methods of the present invention may be determined from biological samples (tissue, blood, urine etc.) taken from a subject prior to, during and subsequent to administration of the AON. Assays of such samples include (1) monitoring the presence or absence of heteroduplex formation with target and non-target sequences, using procedures known to those skilled in the art, e.g., an electrophoretic gel mobility assay; (2) monitoring the amount of a mutant mRNA in relation to a reference normal mRNA or protein as determined by standard techniques such as RT-PCR, Northern blotting, ELISA or Western blotting.

The methods of the invention extends also to a combination of two or more AONs capable of binding to a selected target to induce exon exclusion in a RAGE gene transcript. The combination may be a cocktail of two or more AONs, a construct comprising two or more or two or more AONs joined together for use in an AON-based therapy. The combination of AONs is preferably a combination of SEQ ID NO: 11 and 10, or SEQ ID NO: 11 and 13.

Kits are also provided for use in the methods of the invention, in particular to treat, prevent or ameliorate a disease or condition associated with RAGE expression in a patient, which kit comprises at least an AON as described herein together with instructions for its use, wherein the instructions for use describe a method of the invention.

The contents of the kit can be lyophilized and the kit can additionally contain a suitable solvent for reconstitution of the lyophilized components. Individual components of the kit would be packaged in separate containers and, associated with such containers, can be a notice in the form prescribed by a governmental agency regulating the manufacture, use or sale of pharmaceuticals or biological products, which notice reflects approval by the agency of manufacture, use or sale for human administration.

As used herein the term “derived” and “derived from” shall be taken to indicate that a specific integer may be obtained from a particular source albeit not necessarily directly from that source.

As used herein, the singular forms “a,” “an” and “the” include plural references unless the context clearly dictates otherwise.

Other than in the operating example, or where otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in the specification and claims are approximations that may vary depending upon the desired properties sought to be obtained by the methods of the present invention. Hence “about 80%” means “about 80%” and also “80%”. At the very least, each numerical parameter should be construed in light of the number of significant digits and ordinary rounding approaches.

Notwithstanding that the numerical ranges and parameters setting forth the broad scope of the methods of the present invention are approximations, the numerical values set forth in the specific examples are reported as precisely as possible. Any numerical value; however, inherently contains certain errors necessarily resulting from the standard deviation found in their respective testing measurements

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

It will be understood that the invention disclosed and defined in this specification extends to all alternative combinations of two or more of the individual features mentioned or evident from the text or drawings. All of these different combinations constitute various alternative aspects of the invention.

### EXAMPLES

### Example 1—Materials and Methods

### Animals

Studies undertaken to explore the duration of efficacy of antisense oligonucleotides targeting mouse RAGE pre-RNA were undertaken in healthy pathogen-free male C57BL/6 mice (8-10 weeks old). The animals were housed in sterile passive micro-isolators at a constant 20° C. temperature on a 12-h day/night cycle and fed irradiated Barastoc mouse feed with irradiated tap water allowed ad libitum. Mouse weights were monitored throughout the experiment. Throughout the study animals were given access to mouse chow and water ad libitum. All experiments were approved by local animal ethics committee and conducted in accordance with the Guide for the Care and Use of Laboratory Animals published by the US National Institutes of Health (NIH Publication No. 85-23, revised 1996).

### Intratrachael Treatment

Mice were anaesthetised using a mixture of ketamine/xylazine (90 mg/10 mg/kg body weight via ip injection) and intratracheal treatment undertaken when there was no reflex response (limb withdrawal to the hind paw). The tracheal opening was directly visualised using an otoscope/speculum device placed carefully into the mouth. Fifty microlitres of the allocated sterile solution was administered directly into the trachea through a blunted needle and high pressure syringe. Following the procedure, mice were returned to their home cage to recover on warmed blanket. Mice were monitored until full recovery was observed, after which they were provided fluids in the form of a moistened tissue and moistened food pellets, along with dry food pellets on the floor of the cage and returned to the housing room. Mice were then followed for 3, 7, 14, or 28 days at which time they were humanely killed using CO2 narcosis.

### Microspayer Treatment

Mice were anaesthetised using a mixture of ketamine/xylazine (90 mg/10 mg/kg body weight via ip injection) and intratracheal treatment undertaken when there was no reflex response (limb withdrawal to the hind paw). The tracheal opening was directly visualised using an otoscope/speculum device placed carefully into the mouth. Fifty microlitres of the allocated sterile solution was administered directly into the trachea through an aerosoliser micro-sprayer (Penn-Century IA-1C) connected to a high-pressure syringe (Penn-Century FMJ-250). Between each solution, the microsprayer and syringe was flushed three times with 250 μl of sterile ultrapure water and once with 250 μl sterile saline. Following the procedure, mice were returned to their home cage to recover on paper towel and provided warmth by placing half of the cage on a heat pad. Mice were monitored until full recovery was observed, after which they were provided fluids in the form of a moistened tissue and moistened food pellets, along with dry food pellets on the floor of the cage and returned to the housing room. Mice were then followed for 8-hours, 1, 2, 3, 5, 7, 10, 14, 21 or 28 days at which time they were humanely killed.

### Sample Collection and Harvesting

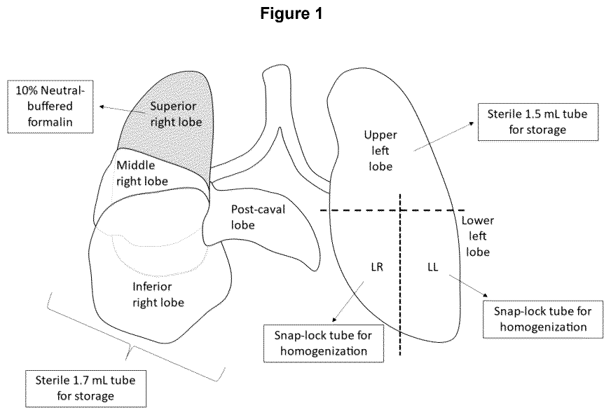

Immediately following their death, all mice were exsanguinated by drawing blood from the inferior vena cava with a 19 G syringe lined with lithium heparin into a Microvette lithium/heparin tube (Sarstedt, Aust.). Blood was maintained at RT for a minimum of 15 minutes and centrifuged at 2000 g for 5 minutes at 20° C. Plasma was transferred into 1.5 mL snap-lock Eppendorf tubes, snap frozen in liquid nitrogen and stored at −80° C. Lungs were perfused to remove circulating blood. A 20 G needle was inserted into the right ventricle and 5-10 mL of chilled, sterile phosphate buffered saline was administered at a rate of 300 μl/second. The lungs were then excised as indicated in FIG. 1. The left lobe was divided into upper, lower left (LL) and lower right (LR) portions and snap frozen in liquid nitrogen.

Measuring Levels of esRAGE in Plasma and Lung Tissue

To measure the effect of interventions on the alternative splicing of RAGE pre-mRNA, the expression of RAGE mRNA splice variants was determined using real-time RT-qPCR, performed using the TaqMan system based on real-time detection of accumulated fluorescence (ABI Prism 7700, Perkin-Elmer Inc, PE Biosystems, Foster City, CA, USA) as previously utilized (43). For in vivo experiments, frozen lung tissue from the left lower lobe was used. RNA extraction cDNA synthesized was performed using the Trizol. Gene expression was estimated by RT-qPCR, performed using either the Taqman or SYBR Green (Sigma) system on the basis of real-time detection of accumulation of fluorescence (Applied Biosystems Q3 and Q5). Gene Expression was normalized to 18S mRNA and reported as fold change compared to the level of expression in control RNA treated cells, which were given an arbitrary value of 1. To define the expression of RAGE mRNA splice variants retaining exon 9b a probe was designed to span exon 9b. To define the expression of RAGE mRNA splice variants retaining exon 10, a PCR primer/probes spanning exon 8-10 were designed. To define the expression of all RAGE mRNA, PCR probe/primers spanning exon 8 and 11 were designed. A reduction in the signal of exon 8-10 of the cytosolic tail relative to exon 8-11 indicates that less full-length RAGE transcript (signalling capable) is produced by the cell.

Levels of murine extracellular soluble RAGE (esRAGE) in plasma and the lower right of the left lower lobe of the lung extracted in RIPA buffer were estimated using a commercial ELISA for murine esRAGE (ELISA Kit-MBS7606654-mybiosource). For lung tissue, results were standardised for protein content using the BCA assay (Pierce) (pg/60 ug protein). Plasma results were standardised to volume (pg/ml plasma).

### Statistical Analysis

Data shown are means±SEM (n=6-8). All datasets were determined to be normally distributed based on previous experiments and were also tested using the Kolmogorov-Smirnov test (a=0.05). Data were analysed using one-way ANOVA with Tukey's multiple comparison test using PRISM software (V6.0d, GraphPad, La Jolla, CA, USA), with P<0.05 considered statistically significant.

### Example 2—Time-Dependent PD Effect of a Single Aerosolised Treatment with ASO2 in Male C57BL/6 Mice

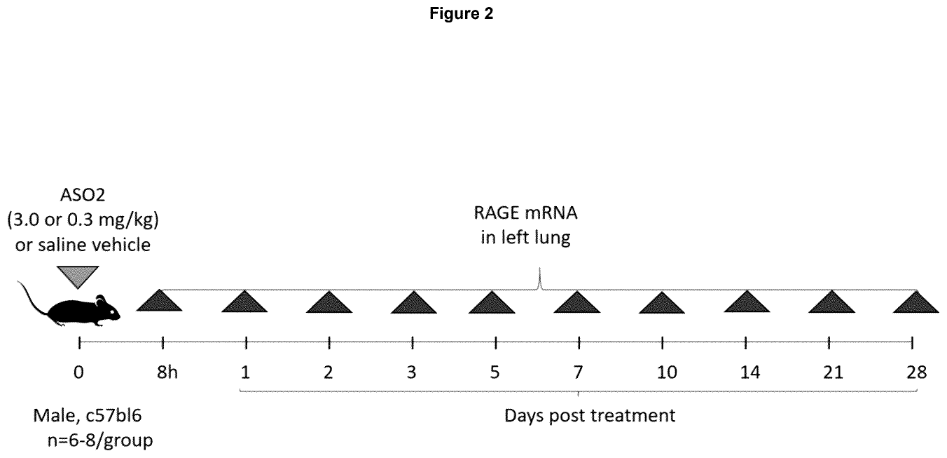

In this experiment, mice were treated with an intratracheal micro-spray of 50 ul of ASO2 (0.3 or 3 mg/kg; 8-10 per group) or saline (vehicle; 4/group). ASO2 is a human 18-mer antisense oligonucleotide with modified nucleotides. ASO2 is an example of an AON that is complementary to a pre-mRNA target region that has a 5′-most nucleotide at position 88 or 114 of exon 10 or that is between nucleotide positions 88 to 114 of exon 10. RAGE_v1 mRNA and esRAGE levels were measured in the lung at various time points (FIG. 2).

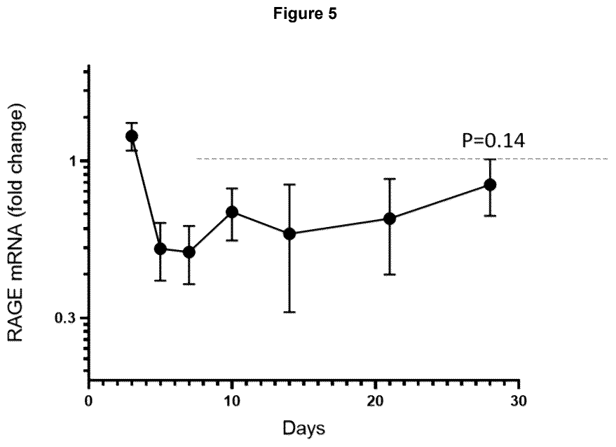

A single dose of ASO2 administered to the lungs resulted in a rapid and sustained induction of RAGE_v1 encoding esRAGE in left lung tissue of mice treated with ASO2 at both 0.3 mg/kg and 3 mg/kg (FIG. 4). No such increase was detected with saline vehicle. ASO2 administration also resulted in a rapid and sustained reduction of RAGE mRNA encoding full length RAGE in the left lung tissue of mice treated with 3 mg/kg compared to vehicle (FIG. 5).

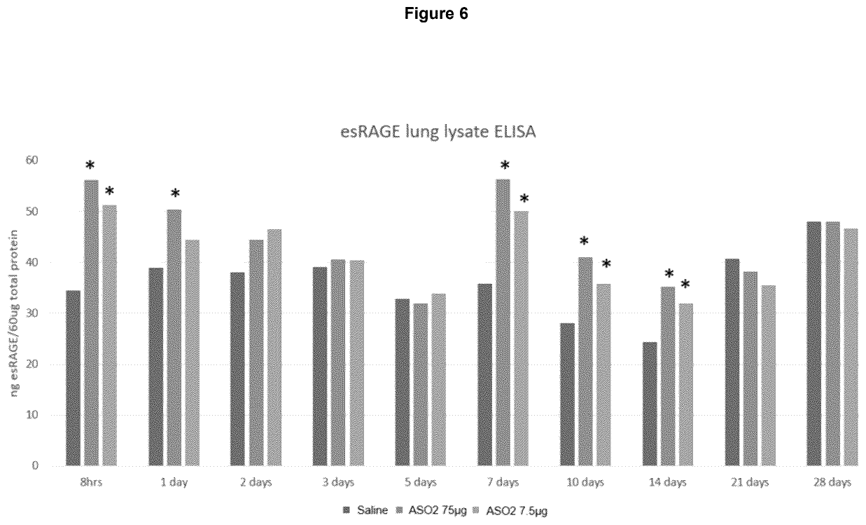

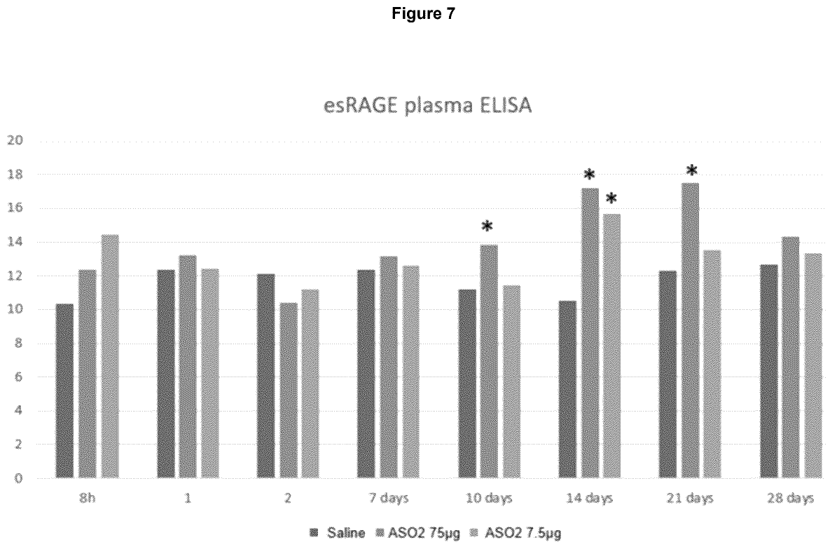

The increase RAGE-v1 mRNA expression correlated with increased esRAGE expression in the lung tissue of the mice treated with both ASO2 at 0.3 mg/kg and 3 mg/kg (about 7.5 μg and 75 μg respectively) at 7, 10 and 14 days compared to control (FIG. 6). Increased esRAGE expression was also detected in plasma at 10, 14 and 21 days following ASO2 treatment (FIG. 7).

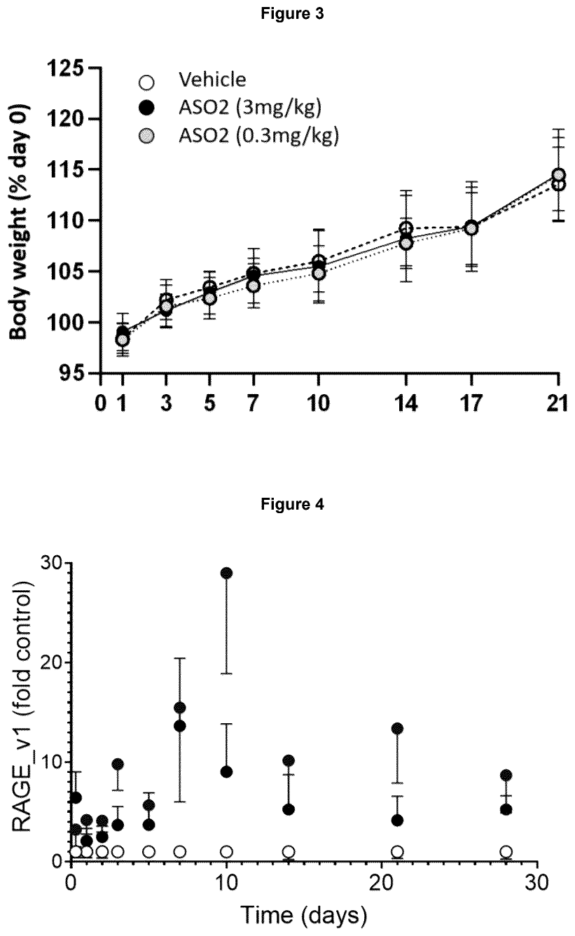

There was no adverse effects on weight gain in individual mice treated with ASO2 when compared to saline vehicle, across a single group of mice (FIG. 3).

### Example 3—Time-Dependent PD Effect of a Single IT Treatment with AON m79 in Male C57BL/6 Mice

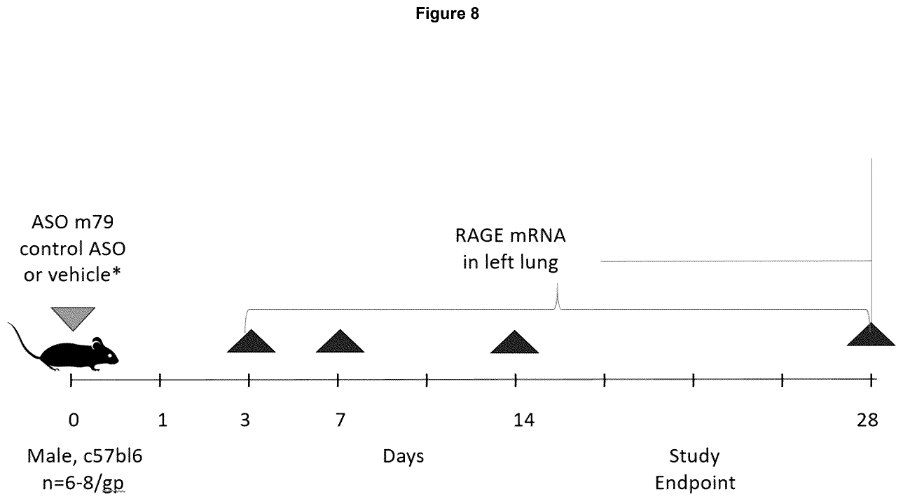

In this experiment, mice (6-8 per group) were treated with an intratracheal micro-spray of 50 ul of AON m79 (3 mg/kg; SEQ NO 27) or a control oligonucleotide (GCAGUUGGCCCCUCCUC; SEQ ID NO: 33) and esRAGE levels were measured in the lung at various time points (FIG. 8). AON m79 is another examples of an AON that is complementary to a pre-mRNA target region that has a 5′-most nucleotide at position 88 or 114 of exon 10 or that is between nucleotide positions 88 to 114 of exon 10.

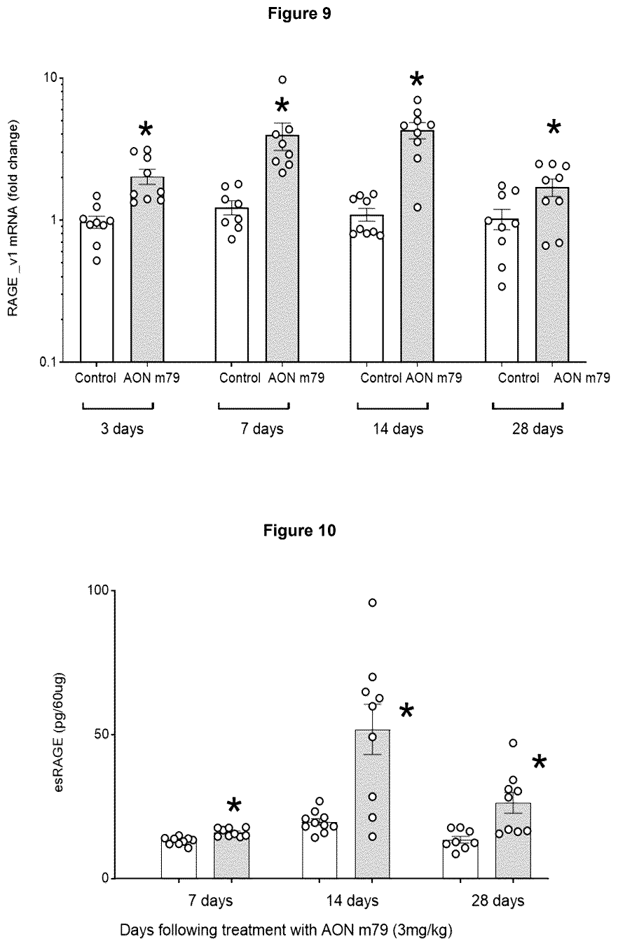

A single dose of AON m79 delivered to the lungs by intratracheal spray resulted in increased RAGE-v1 mRNA expression in lung tissue at 7, 14 and 28 days compared to control (FIG. 9).

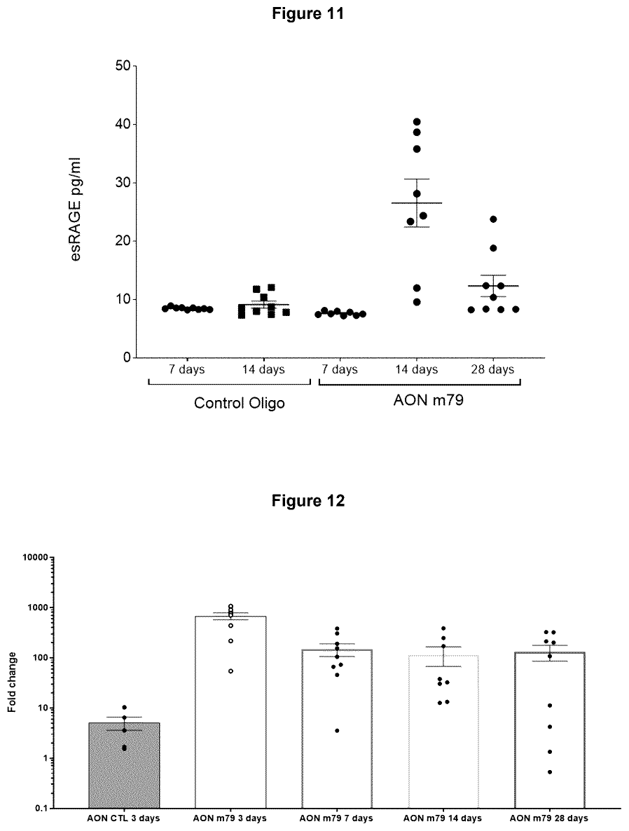

The increase RAGE-v1 mRNA expression correlated with increased esRAGE expression at 7, 14 and 28 days compared to control (FIG. 10), peaking at 14 days. esRAGE was also increased in plasma at 14 and 28 days following the single dose (FIG. 11).

Similarly, AON m79 drug levels were detectable in the left lower lobe of the lung 28 days after a single intratracheal dose with AON m79 (FIG. 12).

This data suggests that single AON dosing can provide an early and persistent increase of esRAGE in lung tissue for up to 28 days following administration. Example 4-Weekly dosing with ASO2 increases RAGE_v1 expression in male C57BL/6 mice

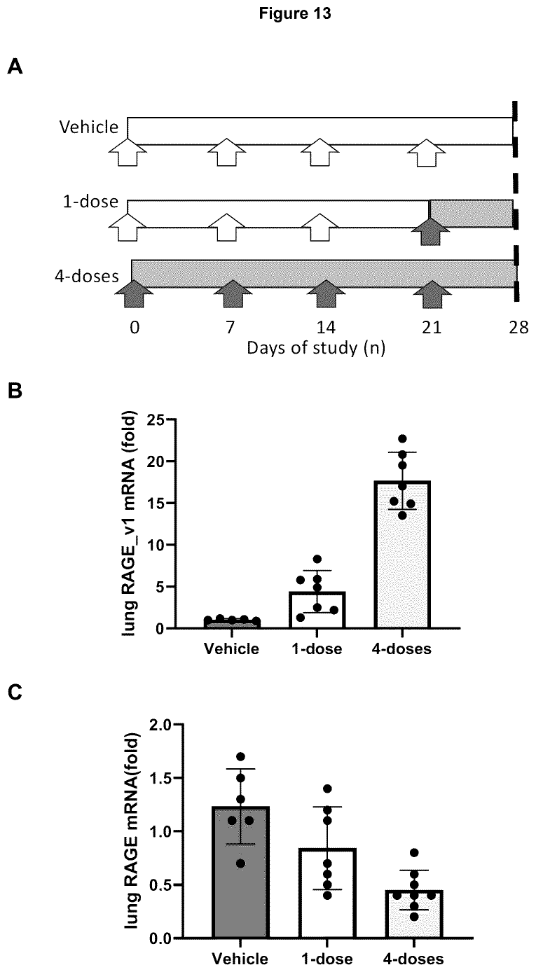

C57BL/6 mice were administered aerosolised saline vehicle weekly for 4 weeks (vehicle group), aerosolised saline vehicle weekly for 3 weeks followed by a single dose of aerosolised ASO2 (3.0 mg/kg) on the 4th week (single dose group), or aerosolised ASO2 (3.0 mg/kg) weekly for 4 weeks (4 dose weekly dosing group) (FIG. 13A).

The 4 dose weekly dosing group resulted in significantly increased RAGE_v1 mRNA expression and decreased RAGE mRNA expression in the lung compared to the single dose or vehicle control groups (FIG. 13B&C). Lung esRAGE was found to be increased and full length RAGE was found decreased in the 4 dose weekly group compared to single dose or vehicle control groups (data not shown).

This data demonstrates that regular weekly AON dosing results in increased RAGE_v1 mRNA expression compared to single dose AON.

1. A method for promoting the production of endogenous soluble RAGE and/or reducing the production of membrane bound RAGE (mRAGE) in a subject, the method comprising:
in a first administration, administering to the respiratory tract of the subject an antisense oligonucleotide (AON) that promotes the production of endogenous soluble RAGE and/or reduces the production of membrane bound RAGE (mRAGE),
in a second administration, administering to the respiratory tract of the subject an AON that promotes the production of endogenous soluble RAGE and/or reduces the production of membrane bound RAGE (mRAGE),
wherein the second administration is at least about 7 days after the first administration,
thereby promoting the production of endogenous soluble RAGE and/or reducing the production of membrane bound RAGE (mRAGE) in the subject.

2. A method for preventing or treating a disease associated with, or caused by, RAGE in a subject, the method comprising:
in a first administration, administering to the respiratory tract of the subject an antisense oligonucleotide (AON) that that promotes the production of endogenous soluble RAGE and/or reduces the production of membrane bound RAGE (mRAGE),
in a second administration, administering to the respiratory tract of the subject AON that promotes the production of endogenous soluble RAGE and/or reduces the production of membrane bound RAGE (mRAGE),
wherein the second administration is at least about 7 days after the first administration,
thereby preventing or treating a disease associated with, or caused by, RAGE in a subject.

3. A method for promoting the production of endogenous soluble RAGE and/or reducing the production of membrane bound RAGE (mRAGE) in a subject, the method comprising:
in a first administration, administering to the respiratory tract of the subject an antisense oligonucleotide (AON) that promotes the production of endogenous soluble RAGE and/or reduces the production of membrane bound RAGE (mRAGE),
in further administrations, administering to the respiratory tract of the subject an AON that promotes the production of endogenous soluble RAGE and/or reduces the production of membrane bound RAGE (mRAGE),
wherein each further administration is at least about 7 days after the previous administration,
thereby promoting the production of endogenous soluble RAGE and/or reducing the production of membrane bound RAGE (mRAGE) in a subject.

4. A method for preventing or treating a disease associated with, or caused by, RAGE in a subject, the method comprising:
in a first administration, administering to the respiratory tract of the subject an antisense oligonucleotide (AON) that promotes the production of endogenous soluble RAGE and/or reduces the production of membrane bound RAGE (mRAGE),
in further administrations, administering to the respiratory tract of the subject an AON that promotes the production of endogenous soluble RAGE and/or reduces the production of membrane bound RAGE (mRAGE),
wherein each further administration is at least about 7 days after the first administration or previous administration,
thereby preventing or treating a disease associated with, or caused by, RAGE in a subject.

5. The method of claim 3 or 4, wherein the further administrations is at least one administration, at least two administrations or at least three administrations.

6. A method for promoting the production of endogenous soluble RAGE and/or reducing the production of membrane bound RAGE (mRAGE) in a subject, the method comprising:
administering to the respiratory tract of a subject, once every at least about 7 days, an antisense oligonucleotide (AON) that promotes the production of endogenous soluble RAGE and/or reduces the production of membrane bound RAGE (mRAGE),
thereby promoting the production of endogenous soluble RAGE and/or reducing the production of membrane bound RAGE (mRAGE) in the subject.

7. A method for preventing or treating a disease associated with, or caused by, RAGE in a subject, the method comprising:
administering to the respiratory tract of a subject, once every at least about 7 days, an antisense oligonucleotide (AON) that promotes the production of endogenous soluble RAGE,
thereby preventing or treating a disease associated with, or caused by, RAGE in a subject.

8. The method of claim 6 or 7, wherein the method comprises administering an AON to the respiratory tract of the subject in a first and second administration; in a first, second and third administration; or in a first, second, third and fourth administration.

9. The method of any one of claims 1 to 8, wherein the method comprises administering an AON to the respiratory tract of the subject once every at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, or at least about 28 days after the first administration.

10. The method of any one of claims 1 to 9, wherein the method promotes the production of endogenous soluble RAGE in the total respiratory tract, lower respiratory tract or upper respiratory tract.

11. The method of claim 10, wherein the method promotes the production of endogenous soluble RAGE and/or reduces the production of membrane bound RAGE (mRAGE) in the lower respiratory tract, preferably the lung.

12. The method of any one of claims 1 to 11, wherein the AON promotes the production of endogenous soluble RAGE and/or reduces the production of membrane bound RAGE (mRAGE) by promoting the inclusion of exon 9b and/or the exclusion (e.g. skipping) of exon 10.

13. The method of any one of claims 1 to 11, wherein AON promotes splicing in the RAGE pre-mRNA resulting in the inclusion of exon 9b and/or skipping of exon 10.

14. The method of claim 12 or 13, wherein administration of the AON results in an increase of the level of RAGE_v1, preferably an increase in the level of RAGE_v1 mRNA in one or more tissues of the respiratory tract.

15. The method of any one of claims 1 to 14, wherein the AON is administered to the total respiratory tract, the upper respiratory tract or the lower respiratory tract.

16. The method of any one of claims 1 to 15, wherein the AON is administered as an aerosol.

17. The method of any one of claims 1 to 16, wherein the AON is administered in a single dose.

18. The method of any one of claims 1 to 17, wherein the AON is administered via the same route of administration in the first, second or further administrations.

19. The method of any one of claims 1 to 18, wherein the same AON is administered in each of the first, second or further administrations.

20. The method of any one of claims 1 to 19, wherein the AON is an AON of 10 to 50 nucleotides comprising a targeting sequence complementary to a region near or within an intron of the RAGE pre-mRNA, optionally, the AON is 10 to 50 nucleotides comprising a targeting sequence complementary or adjacent to a splice site of the RAGE pre-mRNA.

21. The method of any one of claims 1 to 20, wherein, the AON is 10 to 50 nucleotides comprising a targeting sequence complementary to RAGE pre-mRNA which modulates secondary structure of said mRNA to influence splice site selection.

22. The method of any one of claims 1 to 21, wherein the AON is an isolated or purified AON for inducing exclusion (also known as skipping) of one or more exonic sequences in the RAGE gene transcript or part thereof.

23. The method of any one of claims 1 to 22, wherein the AON is an isolated or purified AON for inducing retention of intronic sequences in the RAGE gene transcript or part thereof.

24. The method of any one of claims 1 to 23, wherein the AON comprises at least one modified nucleotide.

25. The method of any one of claims 1 to 24, wherein the AON is chemically-modified to prevent degradation of the pre-mRNA-AON complex, preferably wherein the chemical modification is selected from the group consisting of: phosphorodiamidate morpholino oligomers (PMO), 2′ O-methyl phosphorothioate oligonucleotides (2OMe), and 2′-O-methoxyethyl phosphorothioate oligonucleotides (2′-MOE), locked nucleic acid (LNA) modified AONs, thermostable twisted intercalating nucleic acid (TINA) and peptide nucleic acids (PNAs).

26. The method of any one of claims 1 to 25, wherein the AON is conjugated to moieties to increase its delivery, preferably cell-penetrating peptides (CPPs), vivo-morpholinos (VMO) or peptide phosphorodiamidate morpholino oligomers (PPMO).

27. The method of any one of claims 1 to 26, wherein the AON comprises, consists essentially of or consists of a nucleotide sequence at least 80%, 81%, 82%, 83%, 84%, 85%, 86%, 87%, 88%, 89%, 90%, 91%, 92%, 93%, 94%, 95%, 96%, 97%, 98%, 99% or 100% complementary to a target region of exon 10 of RAGE pre-mRNA over the entirety of the antisense oligonucleotide. Preferably, the 5′-most nucleotide of the target region is nucleotide position 88 or 114 of exon 10 or is between nucleotide positions 88 to 114 of exon 10.

28. The method of any one of claims 1 to 27, wherein the AON is 8 to 40 nucleotides in length, 15 to 25 nucleotides in length or 18 nucleotides in length.

29. The method of any one of claims 1 to 28, wherein the AON is selected from the group comprising the sequences set forth in any of Tables 1a-1d.

30. The method of any one of claims 1 to 29, wherein the AON comprises the nucleotide sequence as set forth in any one of SEQ ID NO: 1-31 or a nucleotide sequence at least 85%, 90% or 95% identical thereto.

31. The method of claim 30, wherein the AON comprises the nucleotide sequence as set forth in SEQ ID NO: 11, 18, 19, or 20, or a nucleotide sequence at least 85%, 90% or 95% identical thereto.
